(19) United States
(12) Patent Application Publication
(10) Pub. No.: US 2024/0254554 A1
(43) Pub. Date: Aug. 1, 2024

(54) Sequencing Chemistries and Encodings for Next-Generation in Situ Sequencing

(71) Applicant: The Board of Trustees of the Leland Stanford Junior University, Stanford, CA (US)

(72) Inventors: Karl A. Deisseroth, Stanford, CA (US); Ethan B. Richman, Palo Alto, CA (US)

(21) Appl. No.: 18/561,475

(22) PCT Filed: May 20, 2022
(86) PCT No.: PCT/US2022/030374

§ 371 Date: Nov. 16, 2023

Related U.S. Application Data
(60) Provisional application No. 63/191,461, filed on May 21, 2021.

(51) Int. Cl. C12Q 1/6874; C12Q 1/6841

(52) U.S. Cl. CPC C12Q 1/6874; C12Q 1/6841

(57) ABSTRACT

Sequencing chemistries and encodings for in situ gene sequencing are provided. Next-generation DNA sequencing and in situ sequencing techniques are dependent on robust, accurate, and efficient read out chemistries and encodings. The sequencing chemistries and encodings provided herein can be used with any commercial sequencing service in which sequencing readout is required. These chemistries and encodings may also be used for non-sequenced detection of target nucleic acids, multiplexed in situ labeling, and various applications in pathology and histology.

### BACKGROUND OF THE INVENTION

Biological samples contain complex and heterogenous genetic information spanning the length scales of individual cells and whole tissues. Spatial patterns of nucleic acids within a cell may reveal properties and abnormalities of cellular function; cumulative distributions of RNA expression may define a cell type or function; and systematic variation in the locations of cell types within a tissue may define tissue function. The combination of anatomical connectivity information encoded in nucleic acids and tissue-wide cell type distributions may span many sections of tissue. Techniques for in situ nucleic acid sequencing must therefore be able to bridge resolutions as small as individual molecules and as large as entire brains. Efficiently collecting and recording this information across orders-of-magnitude differences in lengths requires novel inventions to enhance the robustness, rapidity, automated-, and high throughput-nature of in situ sequencing techniques.

### SUMMARY OF THE INVENTION

Sequencing chemistries and encodings for in situ gene sequencing are provided. Next-generation DNA sequencing and in situ sequencing techniques are dependent on robust, accurate, and efficient read out chemistries and encodings. The sequencing chemistries and encodings provided herein can be used with any commercial sequencing service in which sequencing readout is required. These chemistries and encodings may also be used for non-sequenced detection of target nucleic acids, multiplexed in situ labeling, and various applications in pathology and histology.

In one aspect, a method of sequencing by competitive annealing and ligation to determine a sequence of a target nucleic acid is provided, the method comprising performing one or more sequencing cycles, each cycle comprising: (a) contacting the target nucleic acid with a read oligonucleotide and a set of fluorescently labeled decoding probes, wherein the read oligonucleotide comprises a first complementarity region that is complementary to a reading sequence on the target nucleic acid, and wherein each decoding probe comprises a second complementarity region that is complementary to a probe binding site on the target nucleic acid; (b) ligating the read oligonucleotide to one of the decoding probes of the set of fluorescently labeled decoding probes to generate a fluorescent ligation product, wherein the ligation only occurs when the read oligonucleotide and the decoding probe bind to adjacent sequences on the target nucleic acid and both the read oligonucleotide and the decoding probe have sequences that are exactly complementary to the sequence of the target nucleic acid; (c) removing unligated probes; (d) imaging the fluorescent ligation product to detect the fluorescent label of the decoding probe that ligated to the read oligonucleotide, wherein the fluorescent label identifies a nucleotide of the sequence of the target nucleic acid; and (e) removing the fluorescent ligation product from the target nucleic acid by binding a competitor oligonucleotide to the ligation product, wherein the competitor oligonucleotide comprises a third complementarity region comprising a sequence that is complementary to the reading sequence on the target nucleic acid, wherein the fluorescent ligation product dissociates from the target nucleic acid.

In certain embodiments, the set of fluorescently labeled decoding probes comprises: a first probe encoding a guanine, wherein the first probe comprises a first fluorescent label, a second probe encoding an adenine, wherein the second probe comprises a second fluorescent label, a third probe encoding a cytosine, wherein the third probe comprises a third fluorescent label, and a fourth probe encoding a thymine, wherein the fourth probe comprises a fourth fluorescent label.

In certain embodiments, each fluorescently labeled decoding probe encodes 1 to 3 bases adjacent to a ligation junction where the read oligonucleotide is ligated to the fluorescently labeled decoding probe, wherein fluorescently labeled decoding probes encoding different sequences of bases comprise different fluorescent labels.

In certain embodiments, the read oligonucleotide ranges in length from 8 to 11 nucleotides, including any length within this range such as 8, 9, 10, or 11 nucleotides in length.

In certain embodiments, the read oligonucleotide has a melting temperature ranging from 17° C. to 20° C., including any melting temperature within this range such as 17° C., 18° C., 19° C., or 20° C.

In certain embodiments, the competitor oligonucleotide further comprises a fourth complementarity region comprising a sequence that is complementary to at least a portion of the probe binding site.

In certain embodiments, the fourth complementarity region of the competitor oligonucleotide comprises a sequence that is fully complementary to the entire probe binding site on the target nucleic acid.

In certain embodiments, the competitor oligonucleotide further comprises a fifth complementarity region comprising a sequence that is complementary to a competitor-specific complementary site adjacent to the reading sequence on the target nucleic acid.

In certain embodiments, the competitor-specific complementary site ranges in length from 2 nucleotides to 16 nucleotides.

In certain embodiments, the read oligonucleotide further comprises a competitor-specific complementary sequence.

In certain embodiments, the competitor-specific complementary sequence of the read oligonucleotide ranges in length from 2 nucleotides to 16 nucleotides, including any length within this range such as 2, 3, 4, 5, 6, 7, 8, 9, 10, 11, 12, 13, 14, 15, 16 nucleotides.

In certain embodiments, for sequencing cycles following an initial sequencing cycle, the competitor oligonucleotide used in a previous cycle of sequencing is present during one or more subsequent cycles of sequencing.

In certain embodiments, the sequence of the read oligonucleotide for a current cycle of sequencing is optimized to minimize cross-hybridization with read oligonucleotides for other sequencing cycles.

In certain embodiments, the sequences of the fluorescently labeled decoding probes for a current cycle of sequencing are optimized to minimize cross-hybridization with the fluorescently labeled decoding probes for other sequencing cycles.

In certain embodiments, multiple read oligonucleotides, sets of fluorescently labeled decoding probes, and competitor oligonucleotides having specificity for different target nucleic acids are used to sequence a plurality of different target nucleic acids simultaneously or sequentially.

In certain embodiments, the competitor oligonucleotides remove ligation products from a previous round of sequencing from different target nucleic acids than target nucleic acids currently undergoing steps (a) or (b) of a sequencing cycle.

In certain embodiments, the competitor oligonucleotides remove ligation products from a previous round of sequencing from the same target nucleic acids currently undergoing steps (a) or (b) of a sequencing cycle.

In certain embodiments, the competitor oligonucleotide is a round-specific competitor oligonucleotide comprising a fourth complementarity region comprising a sequence that is complementary to the reading sequence for the next cycle of sequencing.

In certain embodiments, the sequencing reads are in a 5′ to 3′ forward direction or a 3′ to 5′ reverse direction.

In certain embodiments, each fluorescently labeled decoding probe has a fluorophore modification at the 5′ end and each read oligonucleotide has a phosphate at the 5′ end for sequencing reads in the forward direction.

In certain embodiments, each fluorescently labeled decoding probe has a phosphate at the 5′ end and a fluorophore modification at the 3′ end for sequencing reads in the reverse direction.

In certain embodiments, the fluorescent labels are fluorescent dyes or quantum dots.

In certain embodiments, the sequencing is performed with sequential encoding. In some embodiments, each read oligonucleotide comprises a unique sequential orthogonal readout sequence and a unique adjacent competitor-specific complementary sequence for each cycle of sequencing. In some embodiments, the unique sequential orthogonal readout sequence ranges in length from 8 nucleotides to 11 nucleotides, and the unique adjacent competitor-specific complementary sequence ranges in length from 2 nucleotides to 16 nucleotides. In some embodiments, each competitor oligonucleotide comprises a sequence that is complementary to the unique sequential orthogonal readout sequence and the unique adjacent competitor-specific complementary sequence of the read oligonucleotide and at least a portion of the sequence of the fluorescently labeled decoding probe for each cycle of sequencing. In some embodiments, the sequence of the competitor oligonucleotide has partial complementarity or full complementarity to the sequence of the fluorescently labeled decoding probe.

In certain embodiments, sequencing is performed with combinatorial encoding. In some embodiments, multiple read oligonucleotides are used for sequencing, wherein each read oligonucleotide comprises a first complementarity region comprising a combinatorial readout sequence that is complementary to a reading sequence at a separate combinatorial read position on the target nucleic acid, wherein the reading sequence at each separate position on the target nucleic is adjacent to a probe binding site. In some embodiments, each read oligonucleotide further comprises a competitor-specific complementary sequence adjacent to the reading sequence. In some embodiments, the competitor-specific complementary sequence is not complementary to the fluorescently labeled decoding probe. In some embodiments, the reading sequence ranges in length from 8 nucleotides to 11 nucleotides, In some embodiments, the competitor-specific complementary sequence ranges in length from 2 nucleotides to 16 nucleotides. In some embodiments, for each separate combinatorial read position, the competitor oligonucleotide comprises a sequence that is complementary to the combinatorial readout sequence and the competitor-specific complementary sequence of the read oligonucleotide and at least a portion of the sequence of the fluorescently labeled decoding probe for each cycle of sequencing. In some embodiments, the combinatorial encoding uses a hamming code.

In another aspect, a method for in situ gene sequencing of a target nucleic acid in a cell in an intact tissue is provided, the method comprising: (a) contacting a fixed and permeabilized intact tissue with at least a pair of oligonucleotide primers under conditions to allow for specific hybridization, wherein the pair of primers comprise a first oligonucleotide and a second oligonucleotide; wherein each of the first oligonucleotide and the second oligonucleotide comprises a first complementarity region, a second complementarity region sequence, and a third complementarity region; wherein the second oligonucleotide further comprises a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid; (b) adding ligase to ligate the second oligonucleotide and generate a closed nucleic acid circle; (c) performing rolling circle amplification in the presence of a nucleic acid molecule, wherein the performing comprises using the second oligonucleotide as a template and the first oligonucleotide as a primer for a polymerase to form one or more amplicons; (d) embedding the one or more amplicons in the presence of hydrogel subunits to form one or more hydrogel-embedded amplicons; (e) sequencing the one or more amplicons according to a method described herein. In certain embodiments, the sequencing is performed with sequential encoding. In other embodiments, the sequencing is performed with combinatorial encoding.

In another aspect, a method of screening a candidate agent to determine whether the candidate agent modulates gene expression of a nucleic acid in a cell in an intact tissue is provided, the method comprising performing a method described herein to determine the gene sequence of the target nucleic acid in the cell in the intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

In certain embodiments, the detecting comprises performing flow cytometry (e.g., mass cytometry or fluorescence-activated flow cytometry); sequencing; probe binding and electrochemical detection; pH alteration; catalysis induced by enzymes bound to DNA tags; quantum entanglement; Raman spectroscopy; terahertz wave technology; and/or scanning electron microscopy. In some embodiments, the detecting comprises performing microscopy, scanning mass spectrometry, or other imaging techniques

In another aspect, a system is provided, the system comprising: a fluidics device, and a processor unit configured to perform a method described herein. In some embodiments, the system further comprises an imaging chamber. In some embodiments, the system further comprises a pump.

### BRIEF DESCRIPTION OF THE DRAWINGS

FIGS. 1A-1B. Removal of sequencing round signal by competitor hybridization. (FIG. 1A) Increasing the concentration of competitor used for signal removal increases the degree of signal removal over a fixed period of time (2 hours). (FIG. 1B) Quantification of the above.

FIGS. 2A-2C. Reversible chemistry for SEDAL or SCAL. (FIG. 2A) Schematic of a “right” chemistry, in which a read out oligo is ligated at its 5′ to a fluorophore containing oligo, where the 3′ of the fluorophore-containing oligo matches a dibase of a barcode; and a “left” chemistry, in which the order of the oligos is reversed: the read out probe is ligated at its 3′ end to a fluorophore-containing oligo with a 5′ phosphate and a 3′ fluorophore. On the right, a schematic illustrating a sequencing target with multiple distinct read out sites, each of which may be read using right or left chemistries, thus doubling the length of high efficiency reads per read out site. (FIG. 2B) A single cell in a tissue across 6 sequencing rounds using only R (“right”) chemistry. Signal intensities are normalized to the first round. Signal yield decreases across rounds due to greater number of random “N” bases incorporated into the read out oligo across rounds. (FIG. 2C) A single cell in a tissue across 6 sequencing rounds using both R and L (“left”) chemistries. The first 3 sequencing cycles use the R chemistry, and the second 3 use the L chemistry, so that labeling yield remains high across rounds and use of random “N” bases in the read out oligo are minimized.

FIGS. 3A-3B. Competitor hybridization for the removal of sequencing cycle signal. (FIG. 3A) Schematic depiction of the removal of a ligated product by a competitor. Signal is added by the ligation of a two-part oligo C+RO and a fluorophore containing oligo F. C, a round-specific competitor sequence; RO, a round-specific read-out specific sequence; F, a fluorophore-containing oligo. The ligated round label product, C+RO+F anneals to complimentary sites on an amplicon, RO′ and F′, while the C component dangles. Addition of a non-fluorescent set of competitor oligos C′+RO′+F′ specific to the previous round signal displace the labeling strand from the amplicon. (FIG. 3B) Because of the sequence orthogonality between competitors and readout oligos per round, the removal of signal from one round (here, round 2) can be performed simultaneously with the addition of signal for the next round (here, round 3). Left, signal for round 2; right, signal for round 3 with round 2 signal removed.

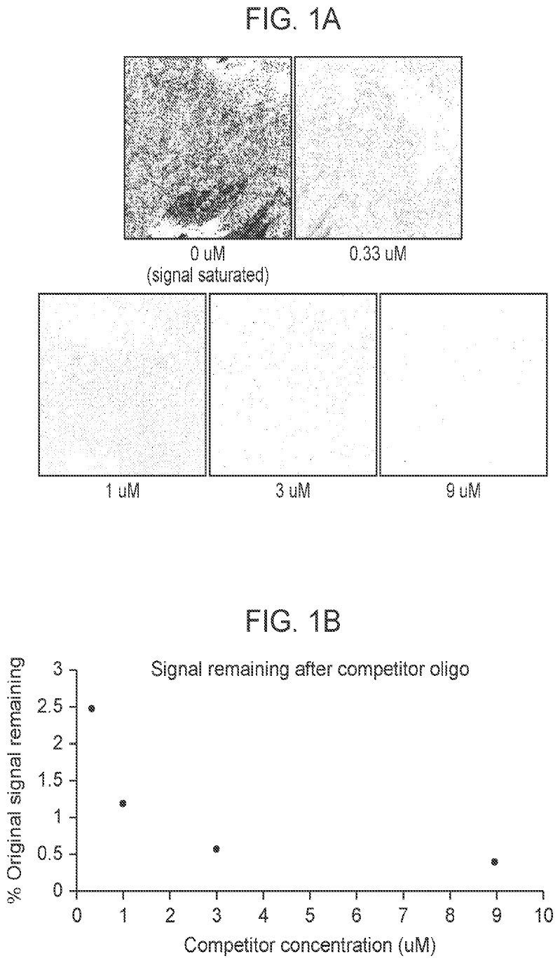

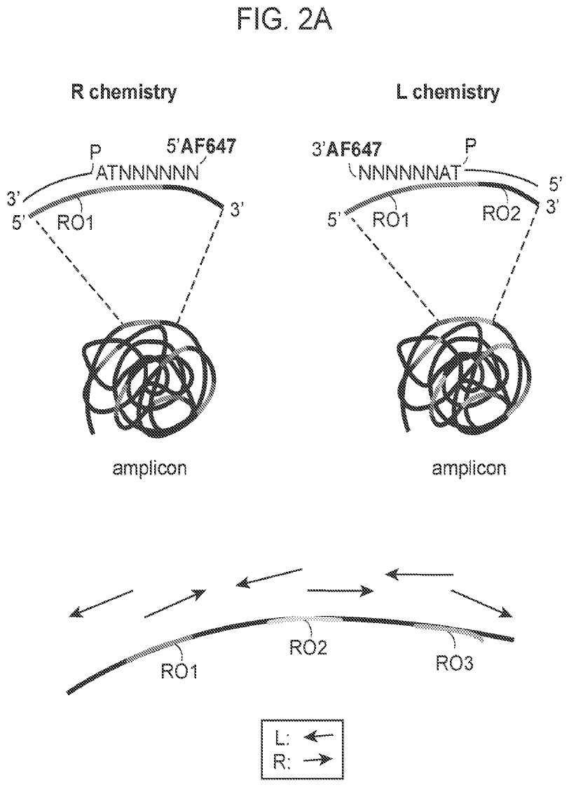

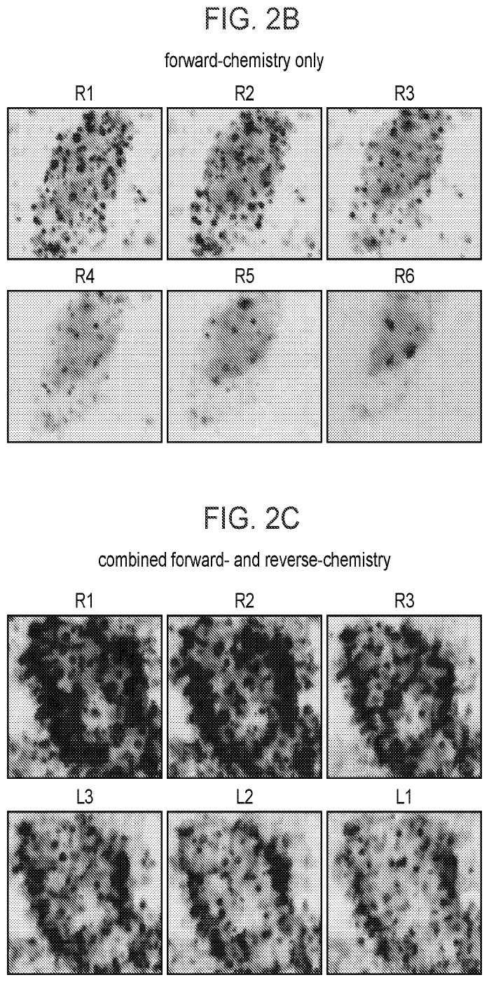

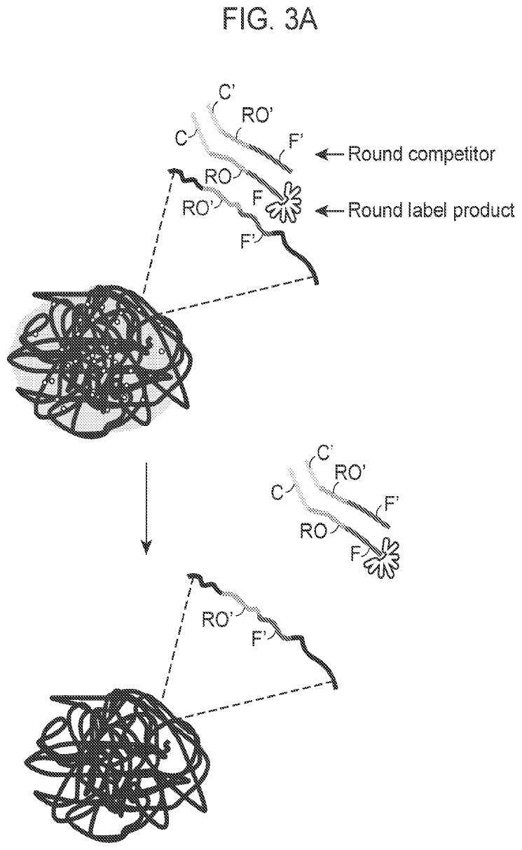

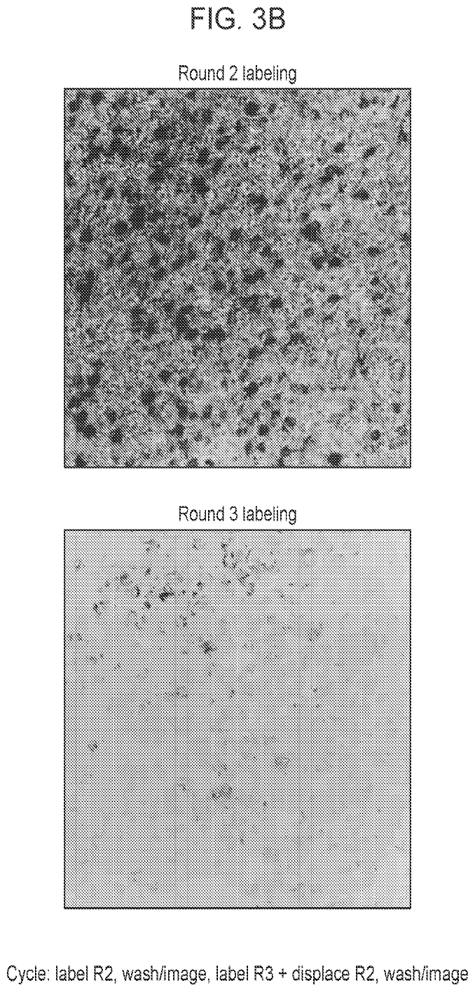

### DETAILED DESCRIPTION OF THE INVENTION

Sequencing chemistries and encodings for in situ gene sequencing are provided. The sequencing chemistries and encodings provided herein can be used with any commercial sequencing service in which sequencing readout is required. These chemistries and encodings may also be used for non-sequenced detection of target nucleic acids, multiplexed in situ labeling, and various applications in pathology and histology.

Before the present sequencing chemistries and encodings are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. oligopeptides or polypeptides known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The terms “peptide”, “oligopeptide”, “polypeptide”, and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers. Both full-length proteins and fragments thereof are encompassed by the definition. The terms also include post-expression modifications of the polypeptide, for example, phosphorylation, glycosylation, acetylation, hydroxylation, oxidation, and the like as well as chemically or biochemically modified or derivatized amino acids and polypeptides having modified peptide backbones. The terms also include fusion proteins, including, but not limited to, fusion proteins with a heterologous amino acid sequence, fusions with heterologous and homologous leader sequences, with or without N-terminal methionine residues; immunologically tagged proteins; and the like. The terms include polypeptides including one or more of a fatty acid moiety, a lipid moiety, a sugar moiety, and a carbohydrate moiety.

As used herein, the term “target nucleic acid” is any polynucleotide nucleic acid molecule (e.g., DNA molecule; RNA molecule, modified nucleic acid, etc.) present in a single cell. In some embodiments, the target nucleic acid is a coding RNA (e.g., mRNA). In some embodiments, the target nucleic acid is a non-coding RNA (e.g., tRNA, rRNA, microRNA (miRNA), mature miRNA, immature miRNA; etc.). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc. Target nucleic acids of interest may be variably expressed, i.e. have a differing abundance, within a cell population, wherein the methods of the invention allow profiling and comparison of the expression levels of nucleic acids, including without limitation RNA transcripts, in individual cells. A target nucleic acid can also be a DNA molecule, e.g. a denatured genomic, viral, plasmid, etc. For example, the methods can be used to detect copy number variants, e.g. in a cancer cell population in which a target nucleic acid is present at different abundance in the genome of cells in the population; a virus-infected cells to determine the virus load and kinetics, and the like.

The terms “oligonucleotide,” “polynucleotide,” and “nucleic acid molecule”, used interchangeably herein, refer to polymeric forms of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. The backbone of the polynucleotide can include sugars and phosphate groups (as may typically be found in RNA or DNA), or modified or substituted sugar or phosphate groups. Alternatively, the backbone of the polynucleotide can include a polymer of synthetic subunits such as phosphoramidites, and/or phosphorothioates, and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. The polynucleotide may include one or more L-nucleosides. A polynucleotide may include modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be modified to include N3′-P5′ (NP) phosphoramidate, morpholino phosphorociamidate (MF), locked nucleic acid (LNA), 2′-O-methoxyethyl (MOE), or 2′-fluoro, arabino-nucleic acid (FANA), which can enhance the resistance of the polynucleotide to nuclease degradation (see, e.g., Faria et al. (2001) Nature Biotechnol. 19:40-44; Toulme (2001) Nature Biotechnol. 19:17-18). A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Immunomodulatory nucleic acid molecules can be provided in various formulations, e.g., in association with liposomes, microencapsulated, etc., as described in more detail herein. A polynucleotide used in amplification is generally single-stranded for maximum efficiency in amplification, but may alternatively be double-stranded. If double-stranded, the polynucleotide can first be treated to separate its strands before being used to prepare extension products. This denaturation step is typically affected by heat, but may alternatively be carried out using alkali, followed by neutralization.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The terms “individual”, “subject”, “host”, and “patient”, are used interchangeably herein and refer to invertebrates and vertebrates including, but not limited to, arthropods (e.g., insects, crustaceans, arachnids), cephalopods (e.g., octopuses, squids), amphibians (e.g., frogs, salamanders, caecilians), fish, reptiles (e.g., turtles, crocodilians, snakes, amphisbaenians, lizards, tuatara), mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese. In some cases, the methods of the invention find use in experimental animals, in veterinary application, and in the development of animal models for disease, including, but not limited to, rodents including mice, rats, and hamsters; primates, and transgenic animals.

### Methods

### Sequencing Chemistries and Encodings for In Situ Gene Sequencing

Sequencing chemistries and encodings for in situ gene sequencing are provided. In one aspect, a method of sequencing by competitive annealing and ligation to determine a sequence of a target nucleic acid is provided, the method comprising performing one or more sequencing cycles, each cycle comprising: (a) contacting the target nucleic acid with a read oligonucleotide and a set of fluorescently labeled decoding probes, wherein the read oligonucleotide comprises a first complementarity region that is complementary to a reading sequence on the target nucleic acid, and wherein each decoding probe comprises a second complementarity region that is complementary to a probe binding site on the target nucleic acid; (b) ligating the read oligonucleotide to one of the decoding probes of the set of fluorescently labeled decoding probes to generate a fluorescent ligation product, wherein the ligation only occurs when the read oligonucleotide and the decoding probe bind to adjacent sequences on the target nucleic acid and both the read oligonucleotide and the decoding probe have sequences that are exactly complementary to the sequence of the target nucleic acid; (c) removing unligated probes; (d) imaging the fluorescent ligation product to detect the fluorescent label of the decoding probe that ligated to the read oligonucleotide, wherein the fluorescent label identifies a nucleotide of the sequence of the target nucleic acid; and (e) removing the fluorescent ligation product from the target nucleic acid by binding a competitor oligonucleotide to the ligation product, wherein the competitor oligonucleotide comprises a third complementarity region comprising a sequence that is complementary to the reading sequence on the target nucleic acid, wherein the fluorescent ligation product dissociates from the target nucleic acid.

In exemplary aspects, the ligation involves each of the read oligonucleotide and a fluorescently labeled decoding probe ligating to form a stable product for imaging only when a perfect match occurs. In certain aspects, the mismatch sensitivity of a ligase enzyme is used to determine the underlying sequence of the target nucleic acid molecule. Inclusion of a polyethylene glycol (PEG) polymer in the sequencing ligation mixture substantially accelerates signal addition onto target nucleic acids. Exemplary PEG polymers have molecular weights ranging from 300 g/mol to 10,000,000 g/mol. In some embodiments, a PEG 6000 polymer is present during ligation of the read oligonucleotide and a fluorescently labeled decoding probe.

In certain embodiments, the set of fluorescently labeled decoding probes comprises: a first probe encoding a guanine, wherein the first probe comprises a first fluorescent label, a second probe encoding an adenine, wherein the second probe comprises a second fluorescent label, a third probe encoding a cytosine, wherein the third probe comprises a third fluorescent label, and a fourth probe encoding a thymine, wherein the fourth probe comprises a fourth fluorescent label.

In certain embodiments, each fluorescently labeled decoding probe encodes 1 to 3 bases adjacent to a ligation junction where the read oligonucleotide is ligated to the fluorescently labeled decoding probe, wherein fluorescently labeled decoding probes encoding different sequences of bases comprise different fluorescent labels.

In certain embodiments, the sequences of the fluorescently labeled decoding probes for a current cycle of sequencing are optimized to minimize cross-hybridization with the fluorescently labeled decoding probes for other sequencing cycles.

In certain embodiments, the read oligonucleotide ranges in length from 8 to 11 nucleotides, including any length within this range such as 8, 9, 10, or 11 nucleotides in length. In some embodiments, the read oligonucleotide has a melting temperature ranging from 17° C. to 20° C., including any melting temperature within this range such as 17° C., 18° C., 19° C., or 20° C.

In certain embodiments, the competitor oligonucleotide further comprises a fourth complementarity region comprising a sequence that is complementary to at least a portion of the probe binding site. In some embodiments, the fourth complementarity region of the competitor oligonucleotide comprises a sequence that is fully complementary to the entire probe binding site on the target nucleic acid. In certain embodiments, the competitor oligonucleotide further comprises a fifth complementarity region comprising a sequence that is complementary to a competitor-specific complementary site adjacent to the reading sequence on the target nucleic acid. In some embodiments, the competitor-specific complementary site ranges in length from 2 nucleotides to 16 nucleotides, including any length within this range such as 2, 3, 4, 5, 6, 7, 8, 9, 10, 11, 12, 13, 14, 15, or 16 nucleotides. In certain embodiments, for sequencing cycles following an initial sequencing cycle, the competitor oligonucleotide used in a previous cycle of sequencing is present during one or more subsequent cycles of sequencing.

In certain embodiments, the read oligonucleotide further comprises a competitor-specific complementary sequence. In some embodiments, the competitor-specific complementary sequence of the read oligonucleotide ranges in length from 2 nucleotides to 16 nucleotides, including any length within this range such as 2, 3, 4, 5, 6, 7, 8, 9, 10, 11, 12, 13, 14, 15, or 16 nucleotides. In certain embodiments, the sequence of the read oligonucleotide for a current cycle of sequencing is optimized to minimize cross-hybridization with read oligonucleotides for other sequencing cycles.

In certain embodiments, multiple read oligonucleotides, sets of fluorescently labeled decoding probes, and competitor oligonucleotides having specificity for different target nucleic acids are used to sequence a plurality of different target nucleic acids simultaneously or sequentially.

In certain embodiments, the competitor oligonucleotides remove ligation products from a previous round of sequencing from different target nucleic acids than target nucleic acids currently undergoing steps (a) or (b) of a sequencing cycle. In certain embodiments, the competitor oligonucleotides remove ligation products from a previous round of sequencing from the same target nucleic acids currently undergoing steps (a) or (b) of a sequencing cycle. In certain embodiments, the competitor oligonucleotide is a round-specific competitor oligonucleotide comprising a fourth complementarity region comprising a sequence that is complementary to the reading sequence for the next cycle of sequencing.

The sequencing reads may be in a 5′ to 3′ forward direction or a 3′ to 5′ reverse direction. For sequencing reads in the forward direction, each fluorescently labeled decoding probe has a fluorophore modification at the 5′ end and each read oligonucleotide has a phosphate at the 5′ end. For sequencing reads in the reverse direction, each fluorescently labeled decoding probe has a phosphate at the 5′ end and a fluorophore modification at the 3′ end.

In certain embodiments, the sequencing is performed with sequential encoding. In some embodiments, each read oligonucleotide comprises a unique sequential orthogonal readout sequence and a unique adjacent competitor-specific complementary sequence for each cycle of sequencing. In some embodiments, the unique sequential orthogonal readout sequence ranges in length from 8 nucleotides to 11 nucleotides, including any length within this range such as 8, 9, 10, or 11 nucleotides. In some embodiments, the unique adjacent competitor-specific complementary sequence ranges in length from 2 nucleotides to 16 nucleotides, including any length within this range such as 2, 3, 4, 5, 6, 7, 8, 9, 10, 11, 12, 13, 14, 15, or 16 nucleotides. In some embodiments, each competitor oligonucleotide comprises a sequence that is complementary to the unique sequential orthogonal readout sequence and the unique adjacent competitor-specific complementary sequence of the read oligonucleotide and at least a portion of the sequence of the fluorescently labeled decoding probe for each cycle of sequencing. In some embodiments, the sequence of the competitor oligonucleotide has partial complementarity or full complementarity to the sequence of the fluorescently labeled decoding probe.

In certain embodiments, sequencing is performed with combinatorial encoding. In some embodiments, multiple read oligonucleotides are used for sequencing, wherein each read oligonucleotide comprises a first complementarity region comprising a combinatorial readout sequence that is complementary to a reading sequence at a separate combinatorial read position on the target nucleic acid, wherein the reading sequence at each separate position on the target nucleic is adjacent to a probe binding site. In some embodiments, each read oligonucleotide further comprises a competitor-specific complementary sequence adjacent to the reading sequence. In some embodiments, the competitor-specific complementary sequence is not complementary to the fluorescently labeled decoding probe. In some embodiments, the reading sequence ranges in length from 8 nucleotides to 11 nucleotides, including any length within this range such as 8, 9, 10, or 11 nucleotides. In some embodiments, the competitor-specific complementary sequence ranges in length from 2 nucleotides to 16 nucleotides, including any length within this range such as 2, 3, 4, 5, 6, 7, 8, 9, 10, 11, 12, 13, 14, 15, or 16 nucleotides. In some embodiments, for each separate combinatorial read position, the competitor oligonucleotide comprises a sequence that is complementary to the combinatorial readout sequence and the competitor-specific complementary sequence of the read oligonucleotide and at least a portion of the sequence of the fluorescently labeled decoding probe for each cycle of sequencing. In some embodiments, the combinatorial encoding uses a hamming code (see Examples).

The sequencing methods described herein can be used for situ gene sequencing of a target nucleic acid in a cell in an intact tissue. In some embodiments, the method of in situ gene sequencing of a target nucleic acid in a cell in an intact tissue comprises: (a) contacting a fixed and permeabilized intact tissue with at least a pair of oligonucleotide primers under conditions to allow for specific hybridization, wherein the pair of primers comprise a first oligonucleotide and a second oligonucleotide; wherein each of the first oligonucleotide and the second oligonucleotide comprises a first complementarity region, a second complementarity region sequence, and a third complementarity region; wherein the second oligonucleotide further comprises a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid; (b) adding ligase to ligate the second oligonucleotide and generate a closed nucleic acid circle; (c) performing rolling circle amplification in the presence of a nucleic acid molecule, wherein the performing comprises using the second oligonucleotide as a template and the first oligonucleotide as a primer for a polymerase to form one or more amplicons; (d) embedding the one or more amplicons in the presence of hydrogel subunits to form one or more hydrogel-embedded amplicons; (e) sequencing the one or more amplicons according to a method described herein. In certain embodiments, the sequencing is performed with sequential encoding. In other embodiments, the sequencing is performed with combinatorial encoding.

In some embodiments, the contacting the one or more hydrogel-embedded amplicons occurs two times or more, including, but not limited to, e.g., three times or more, four times or more, five times or more, six times or more, or seven times or more, eight times or more, nine times or more, ten times or more, eleven times or more, or twelve times or more. In certain embodiments, the contacting the one or more hydrogel-embedded amplicons occurs four times or more for thin tissue specimens. In other embodiments, the contacting the one or more hydrogel-embedded amplicons occurs six times or more for thick tissue specimens. In some embodiments, one or more amplicons can be contacted by a pair of primers for 24 or more hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes. In some embodiments, the methods are performed at room temperature for preservation of tissue morphology with low background noise and error reduction. In some embodiments, the contacting the one or more hydrogel-embedded amplicons includes eliminating error accumulation as sequencing proceeds.

Specimens prepared using the subject methods may be analyzed by any of a number of different types of microscopy, for example, optical microscopy (e.g. bright field, oblique illumination, dark field, phase contrast, differential interference contrast, interference reflection, epifluorescence, confocal, etc., microscopy), laser microscopy, electron microscopy, and scanning probe microscopy. In some aspects, a non-transitory computer readable medium transforms raw images acquired through microscopy of multiple rounds of in situ sequencing first into decoded gene identities and spatial locations and then analyzes the per-cell composition of gene expression.

The term “perfectly matched”, when used in reference to a duplex means that the polynucleotide and/or oligonucleotide strands making up the duplex form a double stranded structure with one another such that every nucleotide in each strand undergoes Watson-Crick base pairing with a nucleotide in the other strand. The term “duplex” includes, but is not limited to, the pairing of nucleoside analogs, such as deoxyinosine, nucleosides with 2-aminopurine bases, peptide nucleic acids (PNAs), and the like, that may be employed. A “mismatch” in a duplex between two oligonucleotides means that a pair of nucleotides in the duplex fails to undergo Watson-Crick bonding.

In some embodiments, the method includes a plurality of read oligonucleotides, including, but not limited to, 5 or more read oligonucleotides, e.g., 8 or more, 10 or more, 12 or more, 15 or more, 18 or more, 20 or more, 25 or more, 30 or more, 35 or more that hybridize to target nucleotide sequences. In some embodiments, a method of the present disclosure includes a plurality of read oligonucleotides, including, but not limited to, 15 or more read oligonucleotides, e.g., 20 or more, 30 or more, 40 or more, 50 or more, 60 or more, 70 or more, and up to 80 different read oligonucleotides that hybridize to 15 or more, e.g., 20 or more, 30 or more, 40 or more, 50 or more, 60 or more, 70 or more, and up to 80 different target nucleotide sequences.

In some embodiments, the methods include a plurality of fluorescently labeled decoding probes, including, but not limited to, 4 or more fluorescently labeled decoding probes, e.g., 8 or more, 10 or more, 12 or more, 16 or more, 18 or more, 20 or more, 25 or more, 30 or more, 35 or more. In some embodiments, a method of the present disclosure includes a plurality of fluorescently labeled decoding probes including, but not limited to, 15 or more fluorescently labeled decoding probes, e.g., 20 or more, 30 or more, 40 or more, 50 or more, 60 or more, 70 or more, and up to 80 different fluorescently labeled decoding probes that hybridize to 15 or more, e.g., 20 or more, 30 or more, 40 or more, 50 or more, 60 or more, 70 or more, and up to 80 different target nucleotide sequences.

A plurality of pairs of oligonucleotide primers can be used in a reaction, where one or more pairs specifically bind to each target nucleic acid. For example, two primer pairs can be used for one target nucleic acid in order to improve sensitivity and reduce variability. It is also of interest to detect a plurality of different target nucleic acids in a cell, e.g. detecting up to 2, up to 3, up to 4, up to 5, up to 6, up to 7, up to 8, up to 9, up to 10, up to 12, up to 15, up to 18, up to 20, up to 25, up to 30, up to 40 or more distinct target nucleic acids.

In certain embodiments, sequencing is performed with a ligase with activity hindered by base mismatches, a read oligonucleotide, and a fluorescently labeled decoding probe. The term “hindered” in this context refers to activity of a ligase that is reduced by approximately 20% or more, such as by 25% or more, such as by 50% or more, such as by 75% or more, such as by 90% or more, such as by 95% or more, such as by 99% or more, such as by 100%. In some embodiments, the third oligonucleotide has a length of 5-15 nucleotides, including, but not limited to, 5-13 nucleotides, 5-10 nucleotides, or 5-8 nucleotides. In some embodiments, the Tₘ of the third oligonucleotide is at room temperature (22-25° C.). In some embodiments, the read oligonucleotide is degenerate, or partially thereof. In some embodiments, the fluorescently labeled decoding probe oligonucleotide has a length of 5-15 nucleotides, including, but not limited to, 5-13 nucleotides, 5-10 nucleotides, or 5-8 nucleotides. In some embodiments, the Tₘ of the fourth oligonucleotide is at room temperature (22°-25° C.). After each cycle of sequencing corresponding to a base readout, the fluorescent ligation product is removed from the target nucleic acid by binding a competitor oligonucleotide to the target nucleic acid, wherein the competitor oligonucleotide comprises a third complementarity region comprising a sequence that is complementary to the reading sequence on the target nucleic acid, wherein the fluorescent ligation product dissociates from the target nucleic acid.

In some embodiments, sequencing involves washing to remove unbound oligonucleotides and unligated probes, thereafter revealing a fluorescent product for imaging. In certain exemplary embodiments, a detectable fluorescent label is used to detect one or more nucleotides and/or oligonucleotides described herein. In certain embodiments, a detectable fluorescent label such as a fluorescent protein, fluorescent dye, or fluorescent quantum dot is used to label probes.

Fluorescent labels and their attachment to nucleotides and/or oligonucleotides are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). Particular methodologies applicable to the invention are disclosed in the following sample of references: U.S. Pat. Nos. 4,757,141, 5,151,507 and 5,091,519. In one aspect, one or more fluorescent dyes are used as labels for labeled target sequences, e.g., as disclosed by U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); Lee et al.; U.S. Pat. No. 5,066,580 (xanthine dyes); U.S. Pat. No. 5,688,648 (energy transfer dyes); and the like. Labelling can also be carried out with quantum dots, as disclosed in the following patents and patent publications: U.S. Pat. Nos. 6,322,901, 6,576,291, 6,423,551, 6,251,303, 6,319,426, 6,426,513, 6,444,143, 5,990,479, 6,207,392, 2002/0045045 and 2003/0017264. As used herein, the term “fluorescent label” includes a signaling moiety that conveys information through the fluorescent absorption and/or emission properties of one or more molecules. Such fluorescent properties include fluorescence intensity, fluorescence lifetime, emission spectrum characteristics, energy transfer, and the like.

Commercially available fluorescent nucleotide analogues readily incorporated into nucleotide and/or oligonucleotide sequences include, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.), ATTO dyes such as ATTO 532, ATTO 550, ATTO 565, ATTO 610, ATTO 620, ATTO 635, ATTO 647, ATTO 655, ATTO 680 and the like. Protocols are known in the art for custom synthesis of nucleotides having other fluorophores (See, Henegariu et al. (2000) Nature Biotechnol. 18:345).

Other fluorophores available for post-synthetic attachment include, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, Cy7 (Amersham Biosciences, Piscataway, N.J.) and the like. FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (610, 647, 680), APC-Alexa dyes and the like.

Examples of fluorescent proteins include, but are not limited to, green fluorescent protein, superfolder green fluorescent protein, enhanced green fluorescent protein, Dronpa (a photoswitchable green fluorescent protein), yellow-green fluorescent protein, yellow fluorescent protein, red fluorescent protein, orange fluorescent protein, blue fluorescent protein, cyan fluorescent protein, violet fluorescent protein, mApple, mNectarine, mNeptune, mCherry, mStrawberry, mPlum, mRaspberry, mCrimson3, mCarmine, mCardinal, mScarlet, mRuby2, FusionRed, mNeonGreen, TagRFP675, and mRFP1. and the like.

Metallic silver or gold particles may be used to enhance signal from fluorescently labeled nucleotide and/or oligonucleotide sequences (Lakowicz et al. (2003) Bio Techniques 34:62).

Biotin, or a derivative thereof, may also be used as a label on a nucleotide and/or an oligonucleotide sequence, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into an oligonucleotide sequence and subsequently coupled to an N-hydroxy succinimide (NHS) derivatized fluorescent dye. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for an oligonucleotide sequence may include fluorescein (FAM), digoxigenin, dinitrophenol (DNP), dansyl, biotin, bromodeoxyuridine (BrdU), hexahistidine (6×His), phosphor-amino acids (e.g. P-tyr, P-ser, P-thr) and the like. In one embodiment the following hapten/antibody pairs are used for detection, in which each of the antibodies is derivatized with a detectable label: biotin/α-biotin, digoxigenin/α-digoxigenin, dinitrophenol (DNP)/α-DNP, 5-Carboxyfluorescein (FAM)/α-FAM.

In certain exemplary embodiments, a nucleotide and/or an oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, PCT publication WO 91/17160 and the like. Many different hapten-capture agent pairs are available for use. Exemplary haptens include, but are not limited to, biotin, des-biotin and other derivatives, dinitrophenol, dansyl, fluorescein, CY5, digoxigenin and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some embodiments, an antioxidant compound is included in the washing and imaging buffers (i.e., “anti-fade buffers”) to reduce photobleaching during fluorescence imaging. Exemplary antioxidants include, without limitation, Trolox (6-hydroxy-2,5,7,8-tetramethylchroman-2-carboxylic acid) and Trolox-quinone, propyl-gallate, tertiary butylhydroquinone, butylated hydroxyanisole, butylated hydroxytoluene, glutathione, ascorbic acid, tocopherols, and glucose with glucose oxidase, and catalase. Such antioxidants have an antifade effect on fluorophores. That is, the antioxidant reduces photobleaching during tiling, greatly enhances the signal-to-noise ratio (SNR) of sensitive fluorophores, and enables higher SNR imaging of thicker samples. For a fixed exposure time, including an antioxidant increases the SNR by increasing the concentration of the non-bleached fluorophore during exposure to light. Including an antioxidant also removes the diminishing returns of longer exposure times (caused by the limited fluorophore lifetime before photobleaching), providing for increased SNR by allowing increased exposure times.

An exemplary sequencing cycle optionally begins with a brief sample wash, before proceeding to the first signal addition. Depending on whether sequential or combinatorial encoding is being used for a particular round, the corresponding set of read oligonucleotides, fluorescently labeled decoding probes, and their round-specific competitors are added and ligated. In combinatorial encodings, the read oligonucleotide for a given position x is added, plus a set of fluorescently labeled dibase-encoding oligonucleotides, plus a competitor oligonucleotide for the previous position that was labeled (unless it is the first round of labeling, in which case competitor oligonucleotide is omitted). In sequential encodings, the read oligonucleotide for a given round x, a 4-channel fluorophore mixture, and a round x−1 competitor oligonucleotide are added, except if it is the first round of labeling. The presence of PEG in the sequencing ligation mixture substantially accelerates the signal addition onto the target. Following incubation of the sample in imaging buffer, the sample is imaged, and briefly rinsed before proceeding to the next sequencing cycle.

The methods disclosed herein also provide for a method of screening a candidate agent to determine whether the candidate agent modulates gene expression of a nucleic acid in a cell in an intact tissue by performing a method described herein to determine the gene sequence of a target nucleic acid in the cell in the intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

In certain aspects, the methods disclosed herein provide for a faster processing time, higher multiplexity, higher efficiency, higher sensitivity, lower error rate, and more spatially resolved cell types, as compared to existing gene expression analysis tools. The methods provide improved sequencing-by-ligation techniques (SCAL and SEDAL2) for in situ sequencing with error reduction. In some other aspects, the methods disclosed herein include spatially sequencing (e.g. reagents, chips or services) for biomedical research and clinical diagnostics (e.g. cancer, bacterial infection, viral infection, etc.) with single-cell and/or single-molecule sensitivity.

### Specific Amplification of Nucleic Acids Via Intramolecular Ligation (SNAIL)

An efficient approach for generating cDNA libraries from cellular RNAs in situ may be utilized, which is referred to herein as SNAIL, for Specific Amplification of Nucleic Acids via Intramolecular Ligation. In certain embodiments, the method includes contacting a fixed and permeabilized intact tissue with at least a pair of oligonucleotide primers under conditions to allow for specific hybridization, wherein the pair of primers includes a first oligonucleotide and a second oligonucleotide.

More generally, the nucleic acid present in a cell of interest in a tissue serves as a scaffold for an assembly of a complex that includes a pair of primers, referred to herein as a first oligonucleotide and a second oligonucleotide. In some embodiments, the contacting the fixed and permeabilized intact tissue includes hybridizing the pair of primers to the same target nucleic acid. In some embodiments, the target nucleic acid is RNA. In such embodiments, the target nucleic acid may be mRNA. In other embodiments, the target nucleic acid is DNA.

As used herein, the terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis. It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

### SNAIL Oligonucleotide Primers

In the subject methods, the SNAIL oligonucleotide primers include at least a first oligonucleotide and a second oligonucleotide; wherein each of the first oligonucleotide and the second oligonucleotide includes a first complementarity region, a second complementarity region, and a third complementarity region; wherein the second oligonucleotide further includes a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, and wherein the first complementarity region of the first oligonucleotide is adjacent to the second complementarity region of the second oligonucleotide. In an alternative embodiment, the second oligonucleotide is a closed circular molecule, and a ligation step is omitted.

The present disclosure provides methods where the contacting a fixed and permeabilized tissue includes hybridizing a plurality of oligonucleotide primers having specificity for different target nucleic acids. In some embodiments, the methods include a plurality of first oligonucleotides, including, but not limited to, 5 or more first oligonucleotides, e.g., 8 or more, 10 or more, 12 or more, 15 or more, 18 or more, 20 or more, 25 or more, 30 or more, 35 or more that hybridize to target nucleotide sequences. In some embodiments, a method of the present disclosure includes a plurality of first oligonucleotides, including, but not limited to, 15 or more first oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. In some embodiments, the methods include a plurality of second oligonucleotides, including, but not limited to, 5 or more second oligonucleotides, e.g., 8 or more, 10 or more, 12 or more, 15 or more, 18 or more, 20 or more, 25 or more, 30 or more, 35 or more. In some embodiments, a method of the present disclosure includes a plurality of second oligonucleotides including, but not limited to, 15 or more second oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. A plurality of oligonucleotide pairs can be used in a reaction, where one or more pairs specifically bind to each target nucleic acid. For example, two primer pairs can be used for one target nucleic acid in order to improve sensitivity and reduce variability. It is also of interest to detect a plurality of different target nucleic acids in a cell, e.g. detecting up to 2, up to 3, up to 4, up to 5, up to 6, up to 7, up to 8, up to 9, up to 10, up to 12, up to 15, up to 18, up to 20, up to 25, up to 30, up to 40 or more distinct target nucleic acids. The primers are typically denatured prior to use, typically by heating to a temperature of at least about 50° C., at least about 60° C., at least about 70° C., at least about 80° C., and up to about 99° C., up to about 95° C., up to about 90° C.

In some embodiments, the primers are denatured by heating before contacting the sample. In certain aspects, the melting temperature (Tₘ) of oligonucleotides is selected to minimize ligation in solution. The “melting temperature” or “Tm” of a nucleic acid is defined as the temperature at which half of the helical structure of the nucleic acid is lost due to heating or other dissociation of the hydrogen bonding between base pairs, for example, by acid or alkali treatment, or the like. The Tₘ of a nucleic acid molecule depends on its length and on its base composition. Nucleic acid molecules rich in GC base pairs have a higher Tₘ than those having an abundance of AT base pairs. Separated complementary strands of nucleic acid spontaneously reassociate or anneal to form duplex nucleic acid when the temperature is lowered below the Tₘ. The highest rate of nucleic acid hybridization occurs approximately 25 degrees C. below the Tₘ. The Tₘ may be estimated using the following relationship: Tₘ=69.3+0.41 (GC) % (Marmur et al. (1962) J. Mol. Biol. 5:109-118).

In certain embodiments, the plurality of second oligonucleotides includes a padlock probe. In some embodiments, the probe includes a detectable label that can be measured and quantitated. The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenicol acetyl transferase, and urease.

In some embodiments, the one or more first oligonucleotides and second oligonucleotides bind to a different region of the target nucleic acid, or target site. In a pair, each target site is different, and the target sites are adjacent sites on the target nucleic acid, e.g. usually not more than 15 nucleotides distant, e.g. not more than 10, 8, 6, 4, or 2 nucleotides distant from the other site, and may be contiguous sites. Target sites are typically present on the same strand of the target nucleic acid in the same orientation. Target sites are also selected to provide a unique binding site, relative to other nucleic acids present in the cell. Each target site is generally from about 19 to about 25 nucleotides in length, e.g. from about 19 to 23 nucleotides, from about 19 to 21 nucleotides, or from about 19 to 20 nucleotides. The pair of first and second oligonucleotides are selected such that each oligonucleotide in the pair has a similar melting temperature for binding to its cognate target site, e.g. the Tₘ may be from about 50° C., from about 52° C., from about 55° C., from about 58°, from about 62° C., from about 65° C., from about 70° C., or from about 72° C. The GC content of the target site is generally selected to be no more than about 20%, no more than about 30%, no more than about 40%, no more than about 50%, no more than about 60%, no more than about 70%,

In some embodiments, the first oligonucleotide includes a first, second, and third complementarity region. The target site of the first oligonucleotide may refer to the first complementarity region. As summarized above, the first complementarity region of the first oligonucleotide may have a length of 19-25 nucleotides. In certain aspects, the second complementarity region of the first oligonucleotide has a length of 3-10 nucleotides, including, e.g., 4-8 nucleotides or 4-7 nucleotides. In some aspects, the second complementarity region of the first oligonucleotide has a length of 6 nucleotides. In some embodiments, the third complementarity region of the first oligonucleotide likewise has a length of 6 nucleotides. In such embodiments, the third complementarity region of the first oligonucleotide has a length of 3-10 nucleotides, including, e.g., 4-8 nucleotides or 4-7 nucleotides.

In some embodiments, second first oligonucleotide includes a first, second, and third complementarity region. The target site of the second oligonucleotide may refer to the second complementarity region. As summarized above, the second complementarity region of the second oligonucleotide may have a length of 19-25 nucleotides. In certain aspects, the first complementarity region of the first oligonucleotide has a length of 3-10 nucleotides, including, e.g., 4-8 nucleotides or 4-7 nucleotides. In some aspects, the first complementarity region of the first oligonucleotide has a length of 6 nucleotides. In some aspects, the first complementarity region of the second oligonucleotide includes the 5′ end of the second oligonucleotide. In some embodiments, the third complementarity region of the second oligonucleotide likewise has a length of 6 nucleotides. In such embodiments, the third complementarity region of the second oligonucleotide has a length of 3-10 nucleotides, including, e.g., 4-8 nucleotides or 4-7 nucleotides. In further embodiments, the third complementarity region of the second oligonucleotide includes the 3′ end of the second oligonucleotide. In some embodiments, the first complementarity region of the second oligonucleotide is adjacent to the third complementarity region of the second oligonucleotide.

In some aspects, the second oligonucleotide includes a barcode sequence, wherein the barcode sequence of the second oligonucleotide provides barcoding information for identification of the target nucleic acid. The term “barcode” refers to a nucleic acid sequence that is used to identify a single cell or a subpopulation of cells. Barcode sequences can be linked to a target nucleic acid of interest during amplification and used to trace back the amplicon to the cell from which the target nucleic acid originated. A barcode sequence can be added to a target nucleic acid of interest during amplification by carrying out amplification with an oligonucleotide that contains a region including the barcode sequence and a region that is complementary to the target nucleic acid such that the barcode sequence is incorporated into the final amplified target nucleic acid product (i.e., amplicon).

### Tissue

As described herein, the methods disclosed include in situ sequencing technology of an intact tissue by at least contact a fixed and permeabilized intact tissue with at least a pair of oligonucleotide primers under conditions to allow for specific hybridization. Tissue specimens suitable for use with the methods described herein generally include any type of tissue specimens collected from living or dead subjects, such as, e.g., biopsy specimens and autopsy specimens, of which include, but are not limited to, epithelium, muscle, connective, and nervous tissue. Tissue specimens may be collected and processed using the methods described herein and subjected to microscopic analysis immediately following processing, or may be preserved and subjected to microscopic analysis at a future time, e.g., after storage for an extended period of time. In some embodiments, the methods described herein may be used to preserve tissue specimens in a stable, accessible and fully intact form for future analysis. In some embodiments, the methods described herein may be used to analyze a previously-preserved or stored tissue specimen. In some embodiments, the intact tissue includes brain tissue such as visual cortex slices. In some embodiments, the intact tissue is a thin slice with a thickness of 5-20 μm, including, but not limited to, e.g., 5-18 μm, 5-15 μm, or 5-10 μm. In other embodiments, the intact tissue is a thick slice with a thickness of 20-200 μm, including, but not limited to, e.g., 50-150 μm, 50-100 μm, or 50-80 μm.

Aspects of the invention include fixing intact tissue. The term “fixing” or “fixation” as used herein is the process of preserving biological material (e.g., tissues, cells, organelles, molecules, etc.) from decay and/or degradation. Fixation may be accomplished using any convenient protocol. Fixation can include contacting the sample with a fixation reagent (i.e., a reagent that contains at least one fixative). Samples can be contacted by a fixation reagent for a wide range of times, which can depend on the temperature, the nature of the sample, and on the fixative(s). For example, a sample can be contacted by a fixation reagent for 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes.

A sample can be contacted by a fixation reagent for a period of time in a range of from 5 minutes to 24 hours, e.g., from 10 minutes to 20 hours, from 10 minutes to 18 hours, from 10 minutes to 12 hours, from 10 minutes to 8 hours, from 10 minutes to 6 hours, from 10 minutes to 4 hours, from 10 minutes to 2 hours, from 15 minutes to 20 hours, from 15 minutes to 18 hours, from 15 minutes to 12 hours, from 15 minutes to 8 hours, from 15 minutes to 6 hours, from 15 minutes to 4 hours, from 15 minutes to 2 hours, from 15 minutes to 1.5 hours, from 15 minutes to 1 hour, from 10 minutes to 30 minutes, from 15 minutes to 30 minutes, from 30 minutes to 2 hours, from 45 minutes to 1.5 hours, or from 55 minutes to 70 minutes.

A sample can be contacted by a fixation reagent at various temperatures, depending on the protocol and the reagent used. For example, in some instances a sample can be contacted by a fixation reagent at a temperature ranging from −22° C. to 55° C., where specific ranges of interest include, but are not limited to 50 to 54° C., 40 to 44° C., 35 to 39° C., 28 to 32° C., 20 to 26° C., 0 to 6° C., and −18 to −22° C. In some instances a sample can be contacted by a fixation reagent at a temperature of −20° C., 4° C., room temperature (22-25° C.), 30° C., 37° C., 42° C., or 52° C.

Any convenient fixation reagent can be used. Common fixation reagents include crosslinking fixatives, precipitating fixatives, oxidizing fixatives, mercurials, and the like. Crosslinking fixatives chemically join two or more molecules by a covalent bond and a wide range of cross-linking reagents can be used. Examples of suitable cross-liking fixatives include but are not limited to aldehydes (e.g., formaldehyde, also commonly referred to as “paraformaldehyde” and “formalin”; glutaraldehyde; etc.), imidoesters, NHS (N-Hydroxysuccinimide) esters, and the like. Examples of suitable precipitating fixatives include but are not limited to alcohols (e.g., methanol, ethanol, etc.), acetone, acetic acid, etc. In some embodiments, the fixative is formaldehyde (i.e., paraformaldehyde or formalin). A suitable final concentration of formaldehyde in a fixation reagent is 0.1 to 10%, 1-8%, 1-4%, 1-2%, 3-5%, or 3.5-4.5%, including about 1.6% for 10 minutes. In some embodiments the sample is fixed in a final concentration of 4% formaldehyde (as diluted from a more concentrated stock solution, e.g., 38%, 37%, 36%, 20%, 18%, 16%, 14%, 10%, 8%, 6%, etc.). In some embodiments the sample is fixed in a final concentration of 10% formaldehyde. In some embodiments the sample is fixed in a final concentration of 1% formaldehyde. In some embodiments, the fixative is glutaraldehyde. A suitable concentration of glutaraldehyde in a fixation reagent is 0.1 to 1%. A fixation reagent can contain more than one fixative in any combination. For example, in some embodiments the sample is contacted with a fixation reagent containing both formaldehyde and glutaraldehyde.

The terms “permeabilization” or “permeabilize” as used herein refer to the process of rendering the cells (cell membranes etc.) of a sample permeable to experimental reagents such as nucleic acid probes, antibodies, chemical substrates, etc. Any convenient method and/or reagent for permeabilization can be used. Suitable permeabilization reagents include detergents (e.g., Saponin, Triton X-100, Tween-20, etc.), organic fixatives (e.g., acetone, methanol, ethanol, etc.), enzymes, etc. Detergents can be used at a range of concentrations. For example, 0.001%-1% detergent, 0.05%-0.5% detergent, or 0.1%-0.3% detergent can be used for permeabilization (e.g., 0.1% Saponin, 0.2% tween-20, 0.1-0.3% triton X-100, etc.). In some embodiments methanol on ice for at least 10 minutes is used to permeabilize.

In some embodiments, the same solution can be used as the fixation reagent and the permeabilization reagent. For example, in some embodiments, the fixation reagent contains 0.1%-10% formaldehyde and 0.001%-1% saponin. In some embodiments, the fixation reagent contains 1% formaldehyde and 0.3% saponin.

A sample can be contacted by a permeabilization reagent for a wide range of times, which can depend on the temperature, the nature of the sample, and on the permeabilization reagent(s). For example, a sample can be contacted by a permeabilization reagent for 24 or more hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes. A sample can be contacted by a permeabilization reagent at various temperatures, depending on the protocol and the reagent used. For example, in some instances a sample can be contacted by a permeabilization reagent at a temperature ranging from −82° C. to 55° C., where specific ranges of interest include, but are not limited to: 50 to 54° C., 40 to 44° C., 35 to 39° C., 28 to 32° C., 20 to 26° C., 0 to 6° C., −18 to −22° C., and −78 to −82° C. In some instances a sample can be contacted by a permeabilization reagent at a temperature of −80° C., −20° C., 4° C., room temperature (22-25° C.), 30° C., 37° C., 42° C., or 52° C.

In some embodiments, a sample is contacted with an enzymatic permeabilization reagent. Enzymatic permeabilization reagents that permeabilize a sample by partially degrading extracellular matrix or surface proteins that hinder the permeation of the sample by assay reagents. Contact with an enzymatic permeabilization reagent can take place at any point after fixation and prior to target detection. In some instances the enzymatic permeabilization reagent is proteinase K, a commercially available enzyme. In such cases, the sample is contacted with proteinase K prior to contact with a post-fixation reagent. Proteinase K treatment (i.e., contact by proteinase K; also commonly referred to as “proteinase K digestion”) can be performed over a range of times at a range of temperatures, over a range of enzyme concentrations that are empirically determined for each cell type or tissue type under investigation. For example, a sample can be contacted by proteinase K for 30 or less minutes, 25 or less minutes, 20 or less minutes, 15 or less minutes, 10 or less minutes, 5 or less minutes, or 2 or less minutes. A sample can be contacted by 1 μg/ml or less, 2 μg/m or less, 4 μg/ml or less, 8 μg/ml or less, 10 μg/ml or less, 20 μg/ml or less, 30 μg/ml or less, 50 μg/ml or less, or 100 μg/ml or less proteinase K. A sample can be contacted by proteinase K at a temperature ranging from 2° C. to 55° C., where specific ranges of interest include, but are not limited to: 50 to 54° C., 40 to 44° C., 35 to 39° C., 28 to 32° C., 20 to 26° C., and 0 to 6° C. In some instances a sample can be contacted by proteinase K at a temperature of 4° C., room temperature (22-25° C.), 30° C., 37° C., 42° C., or 52° C. In some embodiments, a sample is not contacted with an enzymatic permeabilization reagent. In some embodiments, a sample is not contacted with proteinase K. Contact of an intact tissue with at least a fixation reagent and a permeabilization reagent results in the production of a fixed and permeabilized tissue.

### Ligase

In some embodiments, the methods disclosed include adding ligase to ligate the second oligonucleotide and generate a closed nucleic acid circle. In some embodiments, the adding ligase includes adding DNA ligase. In alternative embodiments, the second oligonucleotide is provided as a closed nucleic acid circle, and the step of adding ligase is omitted. In certain embodiments, ligase is an enzyme that facilitates the sequencing of a target nucleic acid molecule.

The term “ligase” as used herein refers to an enzyme that is commonly used to join polynucleotides together or to join the ends of a single polynucleotide. Ligases include ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases include bacterial ligases such as E. coli DNA ligase and Taq DNA ligase, Ampligase® thermostable DNA ligase (Epicentre® Technologies Corp., part of Illumina®, Madison, Wis.) and phage ligases such as T3 DNA ligase, T4 DNA ligase and T7 DNA ligase and mutants thereof.

### Rolling Circle Amplification

In some embodiments, the methods of the invention include the step of performing rolling circle amplification in the presence of a nucleic acid molecule, wherein the performing includes using the second oligonucleotide as a template and the first oligonucleotide as a primer for a polymerase to form one or more amplicons. In such embodiments, a single-stranded, circular polynucleotide template is formed by ligation of the second nucleotide, which circular polynucleotide includes a region that is complementary to the first oligonucleotide. Upon addition of a DNA polymerase in the presence of appropriate dNTP precursors and other cofactors, the first oligonucleotide is elongated by replication of multiple copies of the template. This amplification product can be readily detected by binding to a detection probe. In some embodiments, the polymerase is preincubated without dNTPs to allow the polymerase to penetrate the sample uniformly before performing rolling circle amplification.

In some embodiments, only when a first oligonucleotide and second oligonucleotide hybridize to the same target nucleic acid molecule, the second oligonucleotide can be circularized and rolling-circle amplified to generate a cDNA nanoball (i.e., amplicon) containing multiple copies of the cDNA. The term “amplicon” refers to the amplified nucleic acid product of a PCR reaction or other nucleic acid amplification process. In some embodiments, amine-modified nucleotides are spiked into the rolling circle amplification reaction.

Techniques for rolling circle amplification are known in the art (see, e.g., Baner et al, Nucleic Acids Research, 26:5073-5078, 1998; Lizardi et al, Nature Genetics 19:226, 1998; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). In some embodiments the polymerase is phi29 DNA polymerase.

In certain aspects, the nucleic acid molecule includes an amine-modified nucleotide. In such embodiments, the amine-modified nucleotide includes an acrylic acid N-hydroxysuccinimide moiety modification. Examples of other amine-modified nucleotides include, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

### Amplicon Embedding in a Tissue-Hydrogel Setting

In some embodiments, the methods disclosed include embedding one or more amplicons in the presence of hydrogel subunits to form one or more hydrogel-embedded amplicons. The hydrogel-tissue chemistry described includes covalently attaching nucleic acids to in situ synthesized hydrogel for tissue clearing, enzyme diffusion, and multiple-cycle sequencing while an existing hydrogel-tissue chemistry method cannot. In some embodiments, to enable amplicon embedding in the tissue-hydrogel setting, amine-modified nucleotides are spiked into the rolling circle amplification reaction, functionalized with an acrylamide moiety using acrylic acid N-hydroxysuccinimide esters, and copolymerized with acrylamide monomers to form a hydrogel.

As used herein, the terms “hydrogel” or “hydrogel network” mean a network of polymer chains that are water-insoluble, sometimes found as a colloidal gel in which water is the dispersion medium. In other words, hydrogels are a class of polymeric materials that can absorb large amounts of water without dissolving. Hydrogels can contain over 99% water and may include natural or synthetic polymers, or a combination thereof. Hydrogels also possess a degree of flexibility very similar to natural tissue, due to their significant water content. A detailed description of suitable hydrogels may be found in published U.S. patent application 20100055733, herein specifically incorporated by reference. As used herein, the terms “hydrogel subunits” or “hydrogel precursors” mean hydrophilic monomers, prepolymers, or polymers that can be crosslinked, or “polymerized”, to form a three-dimensional (3D) hydrogel network. Without being bound by any scientific theory, it is believed that this fixation of the biological specimen in the presence of hydrogel subunits crosslinks the components of the specimen to the hydrogel subunits, thereby securing molecular components in place, preserving the tissue architecture and cell morphology.

In some embodiments, the embedding includes copolymerizing the one or more amplicons with acrylamide. As used herein, the term “copolymer” describes a polymer which contains more than one type of subunit. The term encompasses polymer which include two, three, four, five, or six types of subunits.

In certain aspects, the embedding includes clearing the one or more hydrogel-embedded amplicons wherein the target nucleic acid is substantially retained in the one or more hydrogel-embedded amplicons. In such embodiments, the clearing includes substantially removing a plurality of cellular components from the one or more hydrogel-embedded amplicons. In some other embodiments, the clearing includes substantially removing lipids and/or proteins from the one or more hydrogel-embedded amplicons. As used herein, the term “substantially” means that the original amount present in the sample before clearing has been reduced by approximately 70% or more, such as by 75% or more, such as by 80% or more, such as by 85% or more, such as by 90% or more, such as by 95% or more, such as by 99% or more, such as by 100%.

In some embodiments, clearing the hydrogel-embedded amplicons includes performing electrophoresis on the specimen. In some embodiments, the amplicons are electrophoresed using a buffer solution that includes an ionic surfactant. In some embodiments, the ionic surfactant is sodium dodecyl sulfate (SDS). In some embodiments, the specimen is electrophoresed using a voltage ranging from about 10 to about 60 volts. In some embodiments, the specimen is electrophoresed for a period of time ranging from about 15 minutes up to about 10 days. In some embodiments, the methods further involve incubating the cleared specimen in a mounting medium that has a refractive index that matches that of the cleared tissue. In some embodiments, the mounting medium increases the optical clarity of the specimen. In some embodiments, the mounting medium includes glycerol.

### Cells

Methods disclosed herein include a method for in situ gene sequencing of a target nucleic acid in a cell in an intact tissue. In certain embodiments, the cell is present in a population of cells. In certain other embodiments, the population of cells includes a plurality of cell types including, but not limited to, excitatory neurons, inhibitory neurons, and non-neuronal cells. Cells for use in the assays of the invention can be an organism, a single cell type derived from an organism, or can be a mixture of cell types. Included are naturally occurring cells and cell populations, genetically engineered cell lines, cells derived from transgenic animals, etc. Virtually any cell type and size can be accommodated. Suitable cells include bacterial, fungal, plant and animal cells. In one embodiment of the invention, the cells are mammalian cells, e.g. complex cell populations such as naturally occurring tissues, for example blood, liver, pancreas, neural tissue, bone marrow, skin, and the like. Some tissues may be disrupted into a monodisperse suspension. Alternatively, the cells may be a cultured population, e.g. a culture derived from a complex population, a culture derived from a single cell type where the cells have differentiated into multiple lineages, or where the cells are responding differentially to stimulus, and the like.

Cell types that can find use in the subject invention include stem and progenitor cells, e.g. embryonic stem cells, hematopoietic stem cells, mesenchymal stem cells, neural crest cells, etc., endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, ThO T cells, cytotoxic T cells; B cells, pre-B cells, etc.; monocytes; dendritic cells; neutrophils; and macrophages; natural killer cells; mast cells, etc.; adipocytes, cells involved with particular organs, such as thymus, endocrine glands, pancreas, brain, such as neurons, glia, astrocytes, dendrocytes, etc. and genetically modified cells thereof. Hematopoietic cells may be associated with inflammatory processes, autoimmune diseases, etc., endothelial cells, smooth muscle cells, myocardial cells, etc. may be associated with cardiovascular diseases; almost any type of cell may be associated with neoplasias, such as sarcomas, carcinomas and lymphomas; liver diseases with hepatic cells; kidney diseases with kidney cells; etc.

The cells may also be transformed or neoplastic cells of different types, e.g. carcinomas of different cell origins, lymphomas of different cell types, etc. The American Type Culture Collection (Manassas, VA) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCI (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

Cells may be non-adherent, e.g. blood cells including monocytes, T cells, B-cells; tumor cells, etc., or adherent cells, e.g. epithelial cells, endothelial cells, neural cells, etc. In order to profile adherent cells, they may be dissociated from the substrate that they are adhered to, and from other cells, in a manner that maintains their ability to recognize and bind to probe molecules.

Such cells can be acquired from an individual using, e.g., a draw, a lavage, a wash, surgical dissection etc., from a variety of tissues, e.g., blood, marrow, a solid tissue (e.g., a solid tumor), ascites, by a variety of techniques that are known in the art. Cells may be obtained from fixed or unfixed, fresh or frozen, whole or disaggregated samples. Disaggregation of tissue may occur either mechanically or enzymatically using known techniques.

### Imaging

The methods disclosed include imaging the one or more hydrogel-embedded amplicons using any of a number of different types of microscopy, e.g., confocal microscopy, two-photon microscopy, light-field microscopy, intact tissue expansion microscopy, and/or CLARITY™-optimized light sheet microscopy (COLM).

Bright field microscopy is the simplest of all the optical microscopy techniques. Sample illumination is via transmitted white light, i.e. illuminated from below and observed from above. Limitations include low contrast of most biological samples and low apparent resolution due to the blur of out of focus material. The simplicity of the technique and the minimal sample preparation required are significant advantages.

In oblique illumination microscopy, the specimen is illuminated from the side. This gives the image a 3-dimensional appearance and can highlight otherwise invisible features. A more recent technique based on this method is Hoffmann's modulation contrast, a system found on inverted microscopes for use in cell culture. Though oblique illumination suffers from the same limitations as bright field microscopy (low contrast of many biological samples; low apparent resolution due to out of focus objects), it may highlight otherwise invisible structures.

Dark field microscopy is a technique for improving the contrast of unstained, transparent specimens. Dark field illumination uses a carefully aligned light source to minimize the quantity of directly-transmitted (unscattered) light entering the image plane, collecting only the light scattered by the sample. Dark field can dramatically improve image contrast (especially of transparent objects) while requiring little equipment setup or sample preparation. However, the technique suffers from low light intensity in final image of many biological samples, and continues to be affected by low apparent resolution.

Phase contrast is an optical microscopy illumination technique that converts phase shifts in light passing through a transparent specimen to brightness changes in the image. In other words, phase contrast shows differences in refractive index as difference in contrast. The phase shifts themselves are invisible to the human eye, but become visible when they are shown as brightness changes.

In differential interference contrast (DIC) microscopy, differences in optical density will show up as differences in relief. The system consists of a special prism (Nomarski prism, Wollaston prism) in the condenser that splits light in an ordinary and an extraordinary beam. The spatial difference between the two beams is minimal (less than the maximum resolution of the objective). After passage through the specimen, the beams are reunited by a similar prism in the objective. In a homogeneous specimen, there is no difference between the two beams, and no contrast is being generated. However, near a refractive boundary (e.g. a nucleus within the cytoplasm), the difference between the ordinary and the extraordinary beam will generate a relief in the image. Differential interference contrast requires a polarized light source to function; two polarizing filters have to be fitted in the light path, one below the condenser (the polarizer), and the other above the objective (the analyzer).

Another microscopic technique using interference is interference reflection microscopy (also known as reflected interference contrast, or RIC). It is used to examine the adhesion of cells to a glass surface, using polarized light of a narrow range of wavelengths to be reflected whenever there is an interface between two substances with different refractive indices. Whenever a cell is attached to the glass surface, reflected light from the glass and that from the attached cell will interfere. If there is no cell attached to the glass, there will be no interference.

A fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” refers to any microscope that uses fluorescence to generate an image, whether it is a more simple set up like an epifluorescence microscope, or a more complicated design such as a confocal microscope, which uses optical sectioning to get better resolution of the fluorescent image.

Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. COLM provides an alternative microscopy for fast 3D imaging of large clarified samples. COLM interrogates large immunostained tissues, permits increased speed of acquisition and results in a higher quality of generated data.

In single plane illumination microscopy (SPIM), also known as light sheet microscopy, only the fluorophores in the focal plane of the detection objective lens are illuminated. The light sheet is a beam that is collimated in one and focused in the other direction. Since no fluorophores are excited outside the detectors' focal plane, the method also provides intrinsic optical sectioning. Moreover, when compared to conventional microscopy, light sheet methods exhibit reduced photobleaching and lower phototoxicity, and often enable far more scans per specimen. By rotating the specimen, the technique can image virtually any plane with multiple views obtained from different angles. For every angle, however, only a relatively shallow section of the specimen is imaged with high resolution, whereas deeper regions appear increasingly blurred.

Super-resolution microscopy is a form of light microscopy. Due to the diffraction of light, the resolution of conventional light microscopy is limited as stated by Ernst Abbe in 1873. A good approximation of the resolution attainable is the FWHM (full width at half-maximum) of the point spread function, and a precise widefield microscope with high numerical aperture and visible light usually reaches a resolution of −250 nm. Super-resolution techniques allow the capture of images with a higher resolution than the diffraction limit. They fall into two broad categories, “true” super-resolution techniques, which capture information contained in evanescent waves, and “functional” super-resolution techniques, which use experimental techniques and known limitations on the matter being imaged to reconstruct a super-resolution image.

Laser microscopy uses laser illumination sources in various forms of microscopy. For instance, laser microscopy focused on biological applications uses ultrashort pulse lasers, or femtosecond lasers, in a number of techniques including nonlinear microscopy, saturation microscopy, and multiphoton fluorescence microscopy such as two-photon excitation microscopy (a fluorescence imaging technique that allows imaging of living tissue up to a very high depth, e.g. one millimeter)

In electron microscopy (EM), a beam of electrons is used to illuminate a specimen and produce a magnified image. An electron microscope has greater resolving power than a light-powered optical microscope because electrons have wavelengths about 100,000 times shorter than visible light (photons). They can achieve better than 50 μm resolution and magnifications of up to about 10,000,000×whereas ordinary, non-confocal light microscopes are limited by diffraction to about 200 nm resolution and useful magnifications below 2000×. The electron microscope uses electrostatic and electromagnetic “lenses” to control the electron beam and focus it to form an image. These lenses are analogous to but different from the glass lenses of an optical microscope that form a magnified image by focusing light on or through the specimen. Electron microscopes are used to observe a wide range of biological and inorganic specimens including microorganisms, cells, large molecules, biopsy samples, metals, and crystals. Industrially, the electron microscope is often used for quality control and failure analysis. Examples of electron microscopy include Transmission electron microscopy (TEM), Scanning electron microscopy (SEM), reflection electron microscopy (REM), Scanning transmission electron microscopy (STEM) and low-voltage electron microscopy (LVEM).

Scanning probe microscopy (SPM) is a branch of microscopy that forms images of surfaces using a physical probe that scans the specimen. An image of the surface is obtained by mechanically moving the probe in a raster scan of the specimen, line by line, and recording the probe-surface interaction as a function of position. Examples of SPM include atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM).

Intact tissue expansion microscopy (exM) enables imaging of thick preserve specimens with roughly 70 nm lateral resolution. Using ExM the optical diffraction limit is circumvented by physically expanding a biological specimen before imaging, thus bringing sub-diffraction limited structures into the size range viewable by a conventional diffraction-limited microscope. ExM can image biological specimens at the voxel rates of a diffraction limited microscope, but with the voxel sizes of a super-resolution microscope. Expanded samples are transparent, and index-matched to water, as the expanded material is >99% water. Techniques of expansion microscopy are known in the art, e.g., as disclosed in Gao et al., Q&A: Expansion Microscopy, BMC Biol. 2017; 15:50.

### Screening Methods

The methods disclosed herein also provide for a method of screening a candidate agent to determine whether the candidate agent modulates gene expression of a nucleic acid in a cell in an intact tissue. The method comprises performing in situ sequencing, as disclosed herein, to determine the gene sequence of a target nucleic acid in the cell in an intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

In some aspects, the detecting includes performing flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In certain aspects, the flow cytometry is mass cytometry or fluorescence-activated flow cytometry. In some other aspects, the detecting includes performing microscopy, scanning mass spectrometry or other imaging techniques described herein. In such aspects, the detecting includes determining a signal, e.g., a fluorescent signal.

By “test agent,” “candidate agent,” and grammatical equivalents herein, which terms are used interchangeably herein, is meant any molecule (e.g. proteins (which herein includes proteins, polypeptides, and peptides), small (i.e., 5-1000 Da, 100-750 Da, 200-500 Da, or less than 500 Da in size), or organic or inorganic molecules, polysaccharides, polynucleotides, etc.) which are to be tested for activity in a subject assay.

A variety of different candidate agents may be screened by the above methods. Candidate agents encompass numerous chemical classes, e.g., small organic compounds having a molecular weight of more than 50 daltons (e.g., at least about 50 Da, at least about 100 Da, at least about 150 Da, at least about 200 Da, at least about 250 Da, or at least about 500 Da) and less than about 20,000 daltons, less than about 10,000 daltons, less than about 5,000 daltons, or less than about 2,500 daltons. For example, in some embodiments, a suitable candidate agent is an organic compound having a molecular weight in a range of from about 500 Da to about 20,000 Da, e.g., from about 500 Da to about 1000 Da, from about 1000 Da to about 2000 Da, from about 2000 Da to about 2500 Da, from about 2500 Da to about 5000 Da, from about 5000 Da to about 10,000 Da, or from about 10,000 Da to about 20,000 Da.

Candidate agents can include functional groups necessary for structural interaction with proteins, e.g., hydrogen bonding, and can include at least an amine, carbonyl, hydroxyl or carboxyl group, or at least two of the functional chemical groups. The candidate agents can include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In one embodiment, candidate modulators are synthetic compounds. Any number of techniques is available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the candidate agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In one embodiment, candidate agents include proteins (including antibodies, antibody fragments (i.e., a fragment containing an antigen-binding region, single chain antibodies, and the like), nucleic acids, and chemical moieties. In one embodiment, the candidate agents are naturally occurring proteins or fragments of naturally occurring proteins. Thus, for example, cellular extracts containing proteins, or random or directed digests of proteinaceous cellular extracts, may be tested. In this way libraries of prokaryotic and eukaryotic proteins may be made for screening. Other embodiments include libraries of bacterial, fungal, viral, and mammalian proteins (e.g., human proteins).

In one embodiment, the candidate agents are organic moieties. In this embodiment, as is generally described in WO 94/243 14, candidate agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

### Devices and Systems

Also included are devices for performing aspects of the subject methods. The subject devices may include, for example, imaging chambers, electrophoresis apparatus, flow chambers, microscopes, needles, tubing, pumps.

The present disclosure also provides systems for performing the subject methods. Systems may include, e.g. a power supply, a refrigeration unit, waste, a heating unit, a pump, etc. Systems may also include any of the reagents described herein, e.g. imaging buffer, wash buffer, strip buffer, Nissl and DAPI solutions. Systems in accordance with certain embodiments may also include a microscope and/or related imaging equipment, e.g., camera components, digital imaging components and/or image capturing equipment, computer processors configured to collect images according to one or more user inputs, and the like.

As discussed above, the systems described herein include a fluidics device having an imaging chamber and a pump; and a processor unit configured to perform the methods for in situ gene sequencing described herein. In some embodiments, the system enables the automation of in situ sequencing, as described herein, including, but not limited to, (a) contacting a target nucleic acid with a read oligonucleotide and a set of fluorescently labeled decoding probes, wherein the read oligonucleotide comprises a first complementarity region that is complementary to a reading sequence on the target nucleic acid, and wherein each decoding probe comprises a second complementarity region that is complementary to a probe binding site on the target nucleic acid; (b) ligating the read oligonucleotide to one of the decoding probes of the set of fluorescently labeled decoding probes to generate a fluorescent ligation product, wherein the ligation only occurs when the read oligonucleotide and the decoding probe bind to adjacent sequences on the target nucleic acid and both the read oligonucleotide and the decoding probe have sequences that are exactly complementary to the sequence of the target nucleic acid; (c) removing unligated probes; (d) imaging the fluorescent ligation product to detect the fluorescent label of the decoding probe that ligated to the read oligonucleotide, wherein the fluorescent label identifies a nucleotide of the sequence of the target nucleic acid; and (e) removing the fluorescent ligation product from the target nucleic acid by binding a competitor oligonucleotide to the target nucleic acid, wherein the competitor oligonucleotide comprises a third complementarity region comprising a sequence that is complementary to the reading sequence on the target nucleic acid, wherein the fluorescent ligation product dissociates from the target nucleic acid. In some embodiments, the method comprises: (a) contacting a fixed and permeabilized intact tissue with at least a pair of oligonucleotide primers under conditions to allow for specific hybridization, wherein the pair of primers comprise a first oligonucleotide and a second oligonucleotide; wherein each of the first oligonucleotide and the second oligonucleotide comprises a first complementarity region, a second complementarity region sequence, and a third complementarity region; wherein the second oligonucleotide further comprises a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid; (b) adding ligase to ligate the second oligonucleotide and generate a closed nucleic acid circle; (c) performing rolling circle amplification in the presence of a nucleic acid molecule, wherein the performing comprises using the second oligonucleotide as a template and the first oligonucleotide as a primer for a polymerase to form one or more amplicons; (d) embedding the one or more amplicons in the presence of hydrogel subunits to form one or more hydrogel-embedded amplicons; and e) sequencing the one or more amplicons according to a method described herein.

In some embodiments, the system includes an imaging chamber for flowing sequencing chemicals involved in in situ DNA sequencing over a sample. In some embodiments, the system of fluidics and pumps control sequencing chemical delivery to the sample. Buffers may be added/removed/recirculated/replaced by the use of the one or more ports and optionally, tubing, pumps, valves, or any other suitable fluid handling and/or fluid manipulation equipment, for example, tubing that is removably attached or permanently attached to one or more components of a device. For example, a first tube having a first and second end may be attached to a first port and a second tube having a first and second end may be attached to a second port, where the first end of the first tube is attached to the first port and the second end of the first tube is operably linked to a receptacle, e.g. a cooling unit, heating unit, filtration unit, waste receptacle, etc.; and the first end of the second tube is attached to the second port and the second end of the second tube is operably linked to a receptacle, e.g. a cooling unit, beaker on ice, filtration unit, waste receptacle, etc.

In some embodiments, the system includes a non-transitory computer-readable storage medium that has instructions, which when executed by the processor unit, cause the processor unit to control the delivery of chemicals and synchronize this process with a microscope. In some embodiments, the non-transitory computer-readable storage medium includes instructions, which when executed by the processor unit, cause the processor unit to measure an optical signal.

### Utility

Next-generation sequencing and in situ sequencing techniques are all dependent on robust, accurate, and efficient read out chemistries and encodings, which are provided by the methods disclosed herein. The subject methods may be used for any purpose in which sequencing readout is required and may find a number of uses in the art such as in basic research, clinical diagnostics, pathology, and forensics. For example, biomedical research applications include, but are not limited to, spatially resolved gene expression analysis for fundamental biology or drug screening. Clinical diagnostics, applications include, but are not limited to, detecting gene markers such as disease, immune responses, bacterial or viral DNA/RNA for patient samples. Examples of advantages of the methods described herein include efficiency, where it takes merely 3 or 4 days to obtain final data from a raw sample, providing speeds much faster than existing microarray or sequencing technology; highly multiplexed (up to 1000 genes); single-cell and single-molecule sensitivity; preserved tissue morphology; and/or high signal-to-noise ratio with low error rates.

In certain aspects, the subject methods may be applied to the study of molecular-defined cell types and activity-regulated gene expression in mouse visual cortex, and to be scalable to larger 3D tissue blocks to visualize short- and long-range spatial organization of cortical neurons on a volumetric scale not previously accessible. In some embodiments, the methods disclosed herein may be adapted to image DNA-conjugated antibodies for highly multiplexed protein detection.

The devices, methods, and systems of the invention can also be generalized to study a number of heterogeneous cell populations in diverse tissues. Without being bound by any scientific theory, the brain poses special challenges well suited to the sequencing methods described herein. For example, the polymorphic activity-regulated gene (ARG) expression observed across different cell types is likely to depend on both intrinsic cell-biological properties (such as signal transduction pathway-component expression), and on extrinsic properties such as neural circuit anatomy that routes external sensory information to different cells (here in visual cortex). In such cases, in situ transcriptomics can effectively link imaging-based molecular information with anatomical and activity information, thus elucidating brain function and dysfunction.

The devices, methods, and systems disclosed herein enable cellular components, e.g. lipids that normally provide structural support but that hinder visualization of subcellular proteins and molecules to be removed while preserving the 3-dimensional architecture of the cells and tissue because the sample is crosslinked to a hydrogel that physically supports the ultrastructure of the tissue. This removal renders the interior of biological specimen substantially permeable to light and/or macromolecules, allowing the interior of the specimen, e.g. cells and subcellular structures, to be microscopically visualized without time-consuming and disruptive sectioning of the tissue. The procedure is also more rapid than procedures commonly used in the art, as clearance and permeabilization, typically performed in separate steps, may be combined in a single step of removing cellular components. Additionally, the specimen can be iteratively stained, unstained, and re-stained with other reagents for comprehensive analysis. Further functionalization with the polymerizable acrylamide moiety enables amplicons to be covalently anchored within the polyacrylamide network at multiple sites.

In one example, the subject devices, methods, and systems may be employed to evaluate, diagnose or monitor a disease. “Diagnosis” as used herein generally includes a prediction of a subject's susceptibility to a disease or disorder, determination as to whether a subject is presently affected by a disease or disorder, prognosis of a subject affected by a disease or disorder (e.g., identification of cancerous states, stages of cancer, likelihood that a patient will die from the cancer), prediction of a subject's responsiveness to treatment for a disease or disorder (e.g., a positive response, a negative response, no response at all to, e.g., allogeneic hematopoietic stem cell transplantation, chemotherapy, radiation therapy, antibody therapy, small molecule compound therapy) and use of therametrics (e.g., monitoring a subject's condition to provide information as to the effect or efficacy of therapy). For example, a biopsy may be prepared from a cancerous tissue and microscopically analyzed to determine the type of cancer, the extent to which the cancer has developed, whether the cancer will be responsive to therapeutic intervention, etc.

The subject devices, methods, and systems also provide a useful technique for screening candidate therapeutic agents for their effect on a tissue or a disease. For example, a subject, e.g. a mouse, rat, dog, primate, human, etc. may be contacted with a candidate agent, an organ or a biopsy thereof may be prepared by the subject methods, and the prepared specimen microscopically analyzed for one or more cellular or tissue parameters. Parameters are quantifiable components of cells or tissues, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g., mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. Thus, for example, one such method may include detecting cellular viability, tissue vascularization, the presence of immune cell infiltrates, efficacy in altering the progression of the disease, etc. In some embodiments, the screen includes comparing the analyzed parameter(s) to those from a control, or reference, sample, e.g., a specimen similarly prepared from a subject not contacted with the candidate agent. Candidate agents of interest for screening include known and unknown compounds that encompass numerous chemical classes, primarily organic molecules, which may include organometallic molecules, inorganic molecules, genetic sequences, etc. Candidate agents of interest for screening also include nucleic acids, for example, nucleic acids that encode siRNA, shRNA, antisense molecules, or miRNA, or nucleic acids that encode polypeptides. An important aspect of the invention is to evaluate candidate drugs, including toxicity testing; and the like. Evaluations of tissue samples using the subject methods may include, e.g., genetic, transcriptomic, genomic, proteomic, and/or metabolomics analyses.

The subject devices, methods, and systems may also be used to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, ancestry, and the like. Such detection may be used in, for example, diagnosing and monitoring disease as, e.g., described above, in personalized medicine, and in studying paternity.

A database of analytic information can be compiled. These databases may include results from known cell types, references from the analysis of cells treated under particular conditions, and the like. A data matrix may be generated, where each point of the data matrix corresponds to a readout from a cell, where data for each cell may include readouts from multiple labels. The readout may be a mean, median or the variance or other statistically or mathematically derived value associated with the measurement. The output readout information may be further refined by direct comparison with the corresponding reference readout. The absolute values obtained for each output under identical conditions will display a variability that is inherent in live biological systems and also reflects individual cellular variability as well as the variability inherent between individuals.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-85 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of sequencing by competitive annealing and ligation to determine a sequence of a target nucleic acid, the method comprising performing one or more sequencing cycles, each cycle comprising:
(a) contacting the target nucleic acid with a read oligonucleotide and a set of fluorescently labeled decoding probes, wherein the read oligonucleotide comprises a first complementarity region that is complementary to a reading sequence on the target nucleic acid, and wherein each decoding probe comprises a second complementarity region that is complementary to a probe binding site on the target nucleic acid;
(b) ligating the read oligonucleotide to one of the decoding probes of the set of fluorescently labeled decoding probes to generate a fluorescent ligation product, wherein the ligation only occurs when the read oligonucleotide and the decoding probe bind to adjacent sequences on the target nucleic acid and both the read oligonucleotide and the decoding probe have sequences that are exactly complementary to the sequence of the target nucleic acid;
(c) removing unligated probes;
(d) imaging the fluorescent ligation product to detect the fluorescent label of the decoding probe that ligated to the read oligonucleotide, wherein the fluorescent label identifies a nucleotide of the sequence of the target nucleic acid; and
(e) removing the fluorescent ligation product from the target nucleic acid by binding a competitor oligonucleotide to the target nucleic acid, wherein the competitor oligonucleotide comprises a third complementarity region comprising a sequence that is complementary to the reading sequence on the target nucleic acid, wherein the fluorescent ligation product dissociates from the target nucleic acid.

2. The method of aspect 1, wherein the set of fluorescently labeled decoding probes comprises:
a first probe encoding a guanine, wherein the first probe comprises a first fluorescent label,
a second probe encoding an adenine, wherein the second probe comprises a second fluorescent label,
a third probe encoding a cytosine, wherein the third probe comprises a third fluorescent label, and
a fourth probe encoding a thymine, wherein the fourth probe comprises a fourth fluorescent label.

3. The method of aspect 1, wherein each fluorescently labeled decoding probe encodes 1 to 3 bases adjacent to a ligation junction where the read oligonucleotide is ligated to the fluorescently labeled decoding probe, wherein fluorescently labeled decoding probes encoding different sequences of bases comprise different fluorescent labels.

4. The method of aspect 1, wherein the read oligonucleotide ranges in length from 8 to 11 nucleotides.

5. The method of aspect 1, wherein the read oligonucleotide has a melting temperature ranging from 17° C. to 20° C.

6. The method of aspect 1, wherein the competitor oligonucleotide further comprises a fourth complementarity region comprising a sequence that is complementary to at least a portion of the probe binding site.

7. The method of aspect 1, wherein the fourth complementarity region of the competitor oligonucleotide comprises a sequence that is fully complementary to the entire probe binding site on the target nucleic acid.

8. The method of aspect 1, wherein the competitor oligonucleotide further comprises a fifth complementarity region comprising a sequence that is complementary to a competitor-specific complementary site adjacent to the reading sequence on the target nucleic acid.

9. The method of aspect 1, wherein the competitor-specific complementary site ranges in length from 2 nucleotides to 16 nucleotides.

10. The method of aspect 1, wherein the read oligonucleotide further comprises a competitor-specific complementary sequence.

11. The method of aspect 1, wherein the competitor-specific complementary sequence of the read oligonucleotide ranges in length from 2 nucleotides to 16 nucleotides.

12. The method of aspect 1, wherein said removing further comprises washing the target nucleic acid, wherein the ligation product diffuses away from the target nucleic acid.

13. The method of aspect 1, wherein for sequencing cycles following an initial sequencing cycle, the competitor oligonucleotide used in a previous cycle of sequencing is present during one or more subsequent cycles of sequencing.

14. The method of aspect 1 or 2, wherein the sequence of the read oligonucleotide for a current cycle of sequencing is optimized to minimize cross-hybridization with read oligonucleotides for other sequencing cycles.

15. The method of aspect 1 or 2, wherein the sequences of the fluorescently labeled decoding probes for a current cycle of sequencing are optimized to minimize cross-hybridization with the fluorescently labeled decoding probes for other sequencing cycles.

16. The method of aspect 1, wherein multiple read oligonucleotides, sets of fluorescently labeled decoding probes, and competitor oligonucleotides having specificity for different target nucleic acids are used to sequence a plurality of different target nucleic acids simultaneously or sequentially.

17. The method of aspect 3, wherein the competitor oligonucleotides remove ligation products from a previous round of sequencing from different target nucleic acids than target nucleic acids currently undergoing steps (a) or (b) of a sequencing cycle.

18. The method of aspect 3, wherein the competitor oligonucleotides remove ligation products from a previous round of sequencing from the same target nucleic acids currently undergoing steps (a) or (b) of a sequencing cycle.

19. The method of aspect 1, wherein the competitor oligonucleotide is a round-specific competitor oligonucleotide comprising a fourth complementarity region comprising a sequence that is complementary to the reading sequence for the next cycle of sequencing.

20. The method of aspect 1, wherein sequencing reads are in a 5′ to 3′ forward direction or a 3′ to 5′ reverse direction.

21. The method of aspect 7, wherein each fluorescently labeled decoding probe has a fluorophore modification at the 5′ end and each read oligonucleotide has a phosphate at the 5′ end for sequencing reads in the forward direction.

22. The method of aspect 7, wherein each fluorescently labeled decoding probe has a phosphate at the 5′ end and a fluorophore modification at the 3′ end for sequencing reads in the reverse direction.

23. The method of aspect 1, wherein the fluorescent labels are fluorescent dyes or quantum dots.

24. The method of any one of aspects 1-17, wherein sequencing is performed with sequential encoding.

25. The method of aspect 8, wherein each read oligonucleotide comprises a unique sequential orthogonal readout sequence and a unique adjacent competitor-specific complementary sequence for each cycle of sequencing.

26. The method of aspect 9, wherein the unique sequential orthogonal readout sequence ranges in length from 8 nucleotides to 11 nucleotides, and the unique adjacent competitor-specific complementary sequence ranges in length from 2 nucleotides to 16 nucleotides.

27. The method of aspect 9 or 10, wherein each competitor oligonucleotide comprises a sequence that is complementary to the unique sequential orthogonal readout sequence and the unique adjacent competitor-specific complementary sequence of the read oligonucleotide and at least a portion of the sequence of the fluorescently labeled decoding probe for each cycle of sequencing.

28. The method of aspect 9 or 10, wherein the sequence of the competitor oligonucleotide has partial complementarity or full complementarity to the sequence of the fluorescently labeled decoding probe.

29. The method of any one of aspects 1-17, wherein sequencing is performed with combinatorial encoding.

30. The method of aspect 7, wherein multiple read oligonucleotides are used for sequencing, wherein each read oligonucleotide comprises a first complementarity region comprising a combinatorial readout sequence that is complementary to a reading sequence at a separate combinatorial read position on the target nucleic acid, wherein the reading sequence at each separate position on the target nucleic is adjacent to a probe binding site.

31. The method of aspect 7, wherein each read oligonucleotide further comprises a competitor-specific complementary sequence adjacent to the reading sequence.

32. The method of aspect 7, wherein the competitor-specific complementary sequence is not complementary to the fluorescently labeled decoding probe.

33. The method of aspect 9, wherein the reading sequence ranges in length from 8 nucleotides to 11 nucleotides,

34. The method of aspect 9, wherein the competitor-specific complementary sequence ranges in length from 2 nucleotides to 16 nucleotides.

35. The method of aspect 9 or 10, wherein for each separate combinatorial read position, the competitor oligonucleotide comprises a sequence that is complementary to the combinatorial readout sequence and the competitor-specific complementary sequence of the read oligonucleotide and at least a portion of the sequence of the fluorescently labeled decoding probe for each cycle of sequencing.

36. The method of any one of aspects 29-35, wherein the combinatorial encoding uses a hamming code.

37. A method for in situ gene sequencing of a target nucleic acid in a cell in an intact tissue, the method comprising:
(a) contacting a fixed and permeabilized intact tissue with at least a pair of oligonucleotide primers under conditions to allow for specific hybridization, wherein the pair of primers comprise a first oligonucleotide and a second oligonucleotide; wherein each of the first oligonucleotide and the second oligonucleotide comprises a first complementarity region, a second complementarity region sequence, and a third complementarity region; wherein the second oligonucleotide further comprises a barcode sequence;
wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid; (b) adding ligase to ligate the second oligonucleotide and generate a closed nucleic acid circle;
(c) performing rolling circle amplification in the presence of a nucleic acid molecule, wherein the performing comprises using the second oligonucleotide as a template and the first oligonucleotide as a primer for a polymerase to form one or more amplicons;
(d) embedding the one or more amplicons in the presence of hydrogel subunits to form one or more hydrogel-embedded amplicons;
(e) sequencing the one or more amplicons according to the method of any one of aspects 1-36.

38. The method of aspect 37, further comprising preincubating the tissue sample with the polymerase for a sufficient time to allow uniform diffusion of the polymerase throughout the tissue before performing the rolling circle amplification.

39. The method of aspect 37 or 38, wherein said imaging is performed in presence of an anti-fade buffer comprising an antioxidant.

40. The method of aspect 39, wherein the anti-fade buffer comprises Trolox (6-hydroxy-2,5,7,8-tetramethylchroman-2-carboxylic acid) and Trolox-quinone.

41. The method of any one of aspects 37-40, wherein the cell is present in a population of cells.

42. The method of aspect 41, wherein the population of cells comprises a plurality of cell types.

43. The method of any one of aspects 37-42, wherein the contacting the fixed and permeabilized intact tissue comprises hybridizing the primers to the same target nucleic acid.

44. The method of any one of aspects 37-43, wherein the target nucleic acid is RNA or DNA.

45. The method of aspect 44, wherein the RNA is mRNA.

46. The method of any one of aspects 37-45, wherein the second oligonucleotide comprises a padlock probe.

47. The method of any one of aspects 37-46, wherein the first complementarity region of the first oligonucleotide has a length of 19-25 nucleotides.

48. The method of any one of aspects 37-47, wherein the second complementarity region of the first oligonucleotide has a length of 6 nucleotides.

49. The method of any one of aspects 37-48, wherein the third complementarity region of the first oligonucleotide has a length of 6 nucleotides.

50. The method of any one of aspects 37-49, wherein the first complementarity region of the second oligonucleotide has a length of 6 nucleotides.

51. The method of any one of aspects 37-50, wherein the second complementarity region of the second oligonucleotide has a length of 19-25 nucleotides.

52. The method of any one of aspects 37-51, wherein the third complementarity region of the second oligonucleotide has a length of 6 nucleotides.

53. The method of any one of aspects 37-52, wherein the first complementarity region of the second oligonucleotide comprises the 5′ end of the second oligonucleotide.

54. The method of any one of aspects 37-53, wherein the third complementarity region of the second oligonucleotide comprises the 3′ end of the second oligonucleotide.

55. The method of any one of aspects 37-54, wherein the first complementarity region of the second oligonucleotide is adjacent to the third complementarity region of the second oligonucleotide.

56. The method of any one of aspects 37-55, wherein the barcode sequence of the second oligonucleotide provides barcoding information for identification of the target nucleic acid.

57. The method of any one of aspects 37-56, wherein the contacting the fixed and permeabilized intact tissue comprises hybridizing a plurality of oligonucleotide primers having specificity for different target nucleic acids.

58. The method of any one of aspects 37-57, wherein the second oligonucleotide is provided as a closed nucleic acid circle, and the step of adding ligase is omitted.

59. The method of any of aspects 37-58, wherein the melting temperature (Tₘ) of oligonucleotides is selected to minimize ligation in solution.

60. The method of any one of aspects 37-59, wherein the adding ligase comprises adding a DNA ligase.

61. The method of any one of aspects 37-60, wherein the nucleic acid molecule comprises an amine-modified nucleotide.

62. The method of aspect 61, wherein the amine-modified nucleotide comprises an acrylic acid N-hydroxysuccinimide moiety modification.

63. The method of any one of aspects 37-62, wherein the embedding comprises copolymerizing the one or more amplicons with acrylamide.

64. The method of any one of aspects 37-63, wherein the embedding comprises clearing the one or more hydrogel-embedded amplicons wherein the target nucleic acid is substantially retained in the one or more hydrogel-embedded amplicons.

65. The method of aspect 64, wherein the clearing comprises substantially removing a plurality of cellular components from the one or more hydrogel-embedded amplicons.

66. The method of aspect 64 or 65, wherein the clearing comprises substantially removing lipids or proteins, or a combination thereof from the one or more hydrogel-embedded amplicons.

67. The method of any one of aspects 37-66, wherein the contacting the one or more hydrogel-embedded amplicons comprises eliminating error accumulation as sequencing proceeds.

68. The method of any one of aspects 37-67, wherein the imaging comprises imaging the one or more hydrogel-embedded amplicons using confocal microscopy, two-photon microscopy, light-field microscopy, intact tissue expansion microscopy, and/or CLARITY™-optimized light sheet microscopy (COLM).

69. The method of any one of aspects 37-68, wherein the intact tissue is a thin slice.

70. The method of aspect 69, wherein the intact tissue has a thickness of 5-20 μm.

71. The method of aspect 68 or 69, wherein the contacting the one or more hydrogel-embedded amplicons occurs four times or more.

72. The method of any one of aspects 37-71, wherein the intact tissue is a thick slice.

73. The method of aspect 72, wherein the intact tissue has a thickness of 20-200 μm.

74. The method of aspect 71 or 72, wherein the contacting the one or more hydrogel-embedded amplicons occurs six times or more.

75. The method of any one of aspects 37-74, wherein the ligation of the third oligonucleotide and the fourth oligonucleotide is performed in presence of a polyethylene glycol polymer.

76. The method of aspect 75, wherein the PEG polymer is PEG 6000.

77. A method of screening a candidate agent to determine whether the candidate agent modulates gene expression of a nucleic acid in a cell in an intact tissue, the method comprising performing the method of any one of aspects 37-76 to determine the gene sequence of the target nucleic acid in the cell in the intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

78. The method of aspect 77, wherein the detecting comprises performing flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy.

79. The method of aspect 78, wherein the flow cytometry is mass cytometry or fluorescence-activated flow cytometry.

80. The method of any one of aspects 77-79, wherein the detecting comprises performing microscopy, scanning mass spectrometry, or other imaging techniques

81. The method of any one of aspects 77-80, wherein the detecting comprises detecting a signal.

82. The method of aspect 81, wherein the signal is a fluorescent signal.

83. A system, comprising:
a fluidics device, and
a processor unit configured to perform the method of any one of aspects 1-82.

84. The system of aspect 83, further comprising an imaging chamber.

85. The system of aspect 83 or 84, further comprising a pump.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Sequencing Chemistries and Encodings for Next-Generation In Situ Sequencing

### Overview

a. Sequencing by competitive annealing and ligation (SCAL)
   i. Use of competitor strands to remove signal from dynamic annealing and ligation signal addition.
      1. Design of reading sequences with competitor sequences
         a. Competitor sequences to outcompete ligation products from their labeling target
         b. Competitor sequences specific to reading sequences such that previous round signal can be removed simultaneously as current round signal is added
      2. Competitor pools to efficiently remove signal from multicolor sequential encodings or ambiguous/unknown barcode base targets
b. Reversible SCAL and SEDAL2 chemistries
   i. Forward chemistry: 5′ Phosphate on reading oligo and 5′ fluorophore on fluor oligo
   ii. Reverse chemistry: No 5′Phosphate on reading oligo; 5′ Phosphate on fluor oligo and 3′ fluorophore on fluor oligo
   iii. Barcode/encoding sequence on either or both sides of a reading sequence encoding site, maximizing efficiently readable barcode per reading sequence
   iv. Barcode distributed across one or many reading sequence sites, in forward and reverse direction, for arbitrarily long barcodes with high efficiency reads
   v. Error correcting codes such as Hamming to perform error correcting function with SCAL or SEDAL2 chemistry
c. Hyperspectral encodings
   i. Adaptation of SCAL or SEDAL2 fluor oligos to hyperspectral (more than 4 channel) regimes
d. Compressive encodings
   i. Simultaneous detection of multiple encoded positions with SCAL or SEDAL2 (on the same substrate)
      1. Compressive decoding using known codebook to demix simultaneously detected signals per amplicon
   ii. Simultaneous detection of multiple encoded signals on different substrates (compressive labeling)
      1. Non-orthogonal (per signal) encoding exploiting known orthogonality or structure in target expression pattern
   iii. Compressed sensing regime in which multiple of the same encoded signal is mixed across targets in the probe library using a known loading
      1. Subsequent compressed sensing sparse decoding from known loadings

### Detailed Description

### SCAL Sequencing

In Sequencing by Competitive Annealing and Ligation (SCAL), signals are accumulated by dynamic annealing of two oligos (a reading sequencing and a fluorophore sequence) adjacently onto a substrate, subsequent ligation of the two sequences, and removal via competition by a signal-specific competitor oligo with the ligation product-substrate complex. By including a competitor-specific complementary site adjacent to the reading sequence, and by making the competitor complementary both to the competitor-specific sequence, as well as to the reading sequence and some or all of the fluorophore sequence, thermodynamic forces strongly favor competitor-product interactions, which have the effect of dissociating fluorescent ligation product oligos from their substrates and allowing for their removal from the sample by diffusive washing. Because competitor sequences have specificity to reading sequences, they can be used simultaneously with signal addition sequencing reactions for the following sequencing round. For example, while no competitor oligos are present during an initial sequencing round, subsequent sequencing reactions can include competitors for the signal of the previous round, thus dramatically reducing the time and phase complexity of each sequencing cycle. In the case of sequential encodings (see below), orthogonal reading sequences mean that competitors remove ligation products of a previous sequencing round from different substrates than those on which new signal is being accumulated by the current round. In the case of combinatorial encodings (see below), signal removal and addition occur on the same substrate. Round-specific competitor oligos have distinct competitor-specific complementary sites for each round, but share some sequence complementary to the next round's reading sequence for each combinatorial reading site (because round-to-round, signals are read out across adjacent bases, for a given reading site, see below). Thus, when a competitor oligo for a previous round is used simultaneously with signal addition for the current round, a 5-way strand interaction and competition results, which proceeds dynamically but irreversibly as new reading and fluorophore sequences are ligated together, resulting in the removal of the previous round signal from a substrate and the addition of the current round signal onto the same substrate. In some cases, SCAL is performed with competitor sequence for a previously labeled round added in a separate phase from signal addition for a current round.

### Sequencing Encoding—Sequential

Sequential encoding sequences consist of two parts: an orthogonal reading (OR) complementary sequence, which sets the round or sparsity of the read out, and the fluorophore complementary sequence, which encode the four different bases (A, T, C, and G) with four spectrally separable fluorophores (or other numbers of channels, see multi-spectral below). These two parts are placed adjacent to each other on the target, so that annealing of each of the two sequencing oligos in the presence of ligase results in a single ligation product from the two pieces. In SCAL and SEDAL2, the orthogonal reading sequences are 8-11 nucleotides (nt) long and target a melting temperature of up to 17°−20° C. in 330 mM monovalent salt. The sequences are optimized to minimize cross-hybridization between ORs and encodings for other rounds. The fluorophore complementary sequences, unlike in SEDAL sequential encoding, have distinct bases from each other for the three bases at the 3′ end, maximizing the specificity of ligation during sequencing and the hybridization specificity of the fluorophore sequence to its proper target, which has the effect of minimizing cross-talk signal in the fluorescent channels. This is especially important for sequential encoding in which signals are summed per cell as it prevents non-specific labeling signal from exceeding the noise floor.

### Sequencing Encoding—Combinatorial

In SCAL (and in improved encoding schemes for SEDAL2), combinatorial encodings are generated from error robust codebooks, such as a hamming code, subject to constraints on the desired degree of error detection and correction. For instance, a 200-gene set might use a 7-read long hamming code, with minimum hamming distance of 3, yielding one base correction and two error detections. Because sequencing uses a 2-base at a time reading scheme for added ligase specificity, each code sequenced is flanked by known bases (for example, C), such that reads of the codebook are always unambiguous. SCAL and SEDAL2 introduces a reverse chemistry mode in which reading occurs in the opposite direction (3′ to 5′ instead of 5′ to 3′), see below. This allows for the same reading sequence to be used in both forward and reverse directions. Thus, a given reading sequence can be flanked by sequence from the codebook, and codes can be separated across multiple sites. The complete code is reconstructed by reading each base in the code in a prescribed order. A read at a given code position consists of either a forward or reverse reading probe hybridizing directly adjacent to the code read position, and a fluorescently labeled dibase oligo (using, for example, the SOLID encoding scheme and containing 6 additional random N bases) hybridizing to the encoded base and ligating to the reading probe. Because read lengths in a single direction greater than three bases long fall off in efficiency due to exponentially decreasing concentrations of reading oligo for a given code (due to the ambiguous bases at the end of the reading probe), a given encoding is typically separated into islands of 3 code bases flanked by Cs. For instance, a 12-base long code would consist of C, 3 code bases, C, a reading sequence, C, 6 code bases, C, a reading sequence, C, the last 3 code bases, and C. Thus, in SCAL and SEDAL2, two reading sequence sites support a 12-base encoding, read out in two sets of forward reads of 3 bases each and two sets of reverse reads of 3 bases each. Using this encoding mechanism, codes of arbitrary length can be encoded into the oligo, where each read maintains similarly high efficiency and thus reads do not decay in quality across rounds. This allows for the use of highly error correcting codes via excess read length, genome-scale error robust coding capacities, or coding capacities for highly diverse libraries. Furthermore, the addition of multiple read sites facilitates optically sparsified encoding sets, where the total encoding across targets is bucketed, so that only a fraction of the encoded set is read out at a time (for instance, one reading site is used for half of targets and another reading site is used for the other half).

### Compressive Encoding and Decoding (Simultaneous Reads Per Amplicon)

A given code that is read out with SCAL or SEDAL2 may include multiple simultaneous reads from distinct code positions. For instance, two different bases may be read from two different read out sites, or from the same read out site in two different directions, resulting in one or two different fluorescent channels being detected for a given spot simultaneously. A naïve decoding would detect multiple signals per amplicon as a low quality read, but a decoding procedure that jointly estimates amplicon location and barcode identity, from the sparse dictionary of known codes, can be used to enable successful decoding with multiple channel labeling per amplicon (the same decoding procedure can also improve single read decoding over a naïve decoding and error correction/rejection approach). This effectively increases the coding capacity of each round from 4 to 16 channels, or correspondingly halves the number of read out rounds required of a barcode of a given length.

### Compressive Labeling (Overloaded/Multiplexed Read-Out Channels)

In cases where targets with a known expression are labeled, read out rounds may be combined to maximize the number of targets that are labeled in a given round with successful decoding. For example, if two markers strictly separate two categories of cells and can be read out in separable channels, then each category can be labeled with two simultaneous read outs for each orthogonal category. For instance, if excitatory and inhibitory cells have strictly separable markers, and can be identified as such in a given round, then other specific markers for excitatory cell subtypes can be labeled at the same time as inhibitory cell subtypes. Thus, in the ideal case, use of combinatorial marker genes or targets (forming a binary tree) can be used to create compressive sequential read out encoding schemes. This concept can additionally be extended to any structured encoding where the unit of quantification (sum per cell) has orthogonality across units.

In an alternate version of compressive labeling, target encodings (in the probe library) themselves can be overloaded into a single read out/fluorescent oligo channel according to a compressive sensing paradigm, such that a single fluorescent channel on a given round contains signal from multiple, potentially overlapping targets, and can be demixed via the known loadings in the encoding.

### Hyperspectral Encoding

Fluorescent oligos are not limited to encoding only 4 color channels. As ligase specificity extends to 2-3 bases adjacent to the ligation junction, each additional differentiating base used increases the channel coding capacity by up to 4 times. Thus, fluorescent oligos that differed from each other by 1 to 2 bases at the ligated end could be used to encode up to 16 different spectral channels; oligos that differed from each other by up to 3 bases at the end could be used to encode up to 64 different spectral channels. To achieve this many different channels, other emitters besides dyes could be used, including quantum dots or other molecules with tunable spectral signatures.

### Sequential Sequencing Oligos

Sequential (orthogonal) readout sequences (OR) were designed with sequential encodings (see above, 8-11 nt orthogonal set), plus an adjacent competitor-specific complementary sequences between 2 and 16 nt in length (typically 16 nt). Competitor-specific complementary sequences do not complement the labeling target/substrate, while the read-out sequence does. Each round has both a unique competitor-specific complementary sequencing and a unique, orthogonal readout sequence. Forward chemistry sequential read outs contain a 5′-phosphate; reverse chemistry sequential read outs do not contain any modifications.

Fluorescent oligo sequences for sequential sequencing are one of 4 different fluorophore-modified oligos (in the case of 4-color imaging), that are especially distinct from each other at the end of the oligo to confer both hybridization and ligation specificity. Except in cases where sequential sequencing oligos contain “N” bases, one of four (when 4 detection color channels are used) sequential sequencing oligos typically are exactly complementary to sequence encoded on the target. When used in forward chemistry, the fluorescent oligo has a fluorophore modification at the 5′ end; when used in reverse chemistry, the fluorescent oligo sequence has a 5′ Phosphate and a fluorophore modification at the 3′ end. See hyperspectral encoding for cases in which more than 4 detection channels are used for encoding.

### Sequential Sequencing Competitors

Sequential sequencing competitor oligos were designed such that each orthogonal reading oligo has a corresponding set of competitor oligos. A corresponding set of competitor oligos complements a given orthogonal reading oligo's competitor-specific complementary sequence (2-16 nt in length, see sequential sequencing oligos), and is followed on the competitor oligo by sequence complementary to the orthogonal reading sequence (8-11 nt in length, see sequential sequencing oligos). Following this, some or all of each of the four fluorescent oligo sequence is complemented on each corresponding competitor in the competitor set for a given orthogonal reading oligo (2-8 nt). The component complementary to the fluorescent oligo maybe fully or partially complemented; partial complementarity allows higher concentration of competitor oligo to be used during the signal addition phase for the subsequent round (as fluorescent oligos are not round specific and are thus also complementary to the competitor oligo from the previous sequencing round, for example). The result is that at least one of the competitor oligo set forms a continuous or largely continuous hairpin along the length of the corresponding reading oligo—fluorescent oligo ligation product, thus displacing it from the target it was labeling previously. In the case of hyperspectral encodings, or fluorophore encodings with unknown bases (see combinatorial sequencing oligos/competitors below), the component of the competitor that is complementary to the fluorescent oligo can simply be “N” bases (a competitor pool specific to a given orthogonal reading oligo).

### Combinatorial Sequencing Oligos

Combinatorial sequencing reading oligos (RO) (which set the round/sparsity/position) of a given read are complementary to the target substrate at a particular position, such that a separate fluorescent oligo can anneal directly adjacently and be ligated together with the RO in the presence of ligase. Reading oligo sequence complementary to the target is between 8-11 nt in length. RO sequence also consists of additional competitor-specific complementary sequence (which does not complement the labeling target) adjacent to the target complementary sequence and is between 2-16 nt in length. When reading a combinatorial barcode, the first two unknown bases of the barcode (adjacent to the reading sequence) are positioned using known reading sequence bases; the third unknown base of the barcode is positioned using an additional “N” base at the end of the reading oligo, and subsequent positions (if more than 3 bases are read from a given read out site) are read via additional N bases at the reading oligo end. In the case of forward read chemistry, the reading oligo is at the 5′ end and the oligo has a 5′ phosphate modification. In the case of reverse read chemistry, the reading oligo is at the 3′ end and there is no phosphate modification.

Fluorescent oligos for combinatorial sequencing, when using 4 channel, 16-oligo SOLID encoding, consist of a pool of 16 oligos with 6 or more N bases and two known bases adjacent to the ligation junction (at the end of the oligo), with fluorophore at the opposite end of the oligo from the known bases to set the corresponding encoding. In the case of forward read chemistry, the fluorescent oligos have a 5′ fluorescent modification and no phosphate modification; in the case of the reverse chemistry, the fluorescent oligos have a 5′ phosphate modification and a 3′ fluorescent modification.

Other fluorescent encoding schemes are also possible with this chemistry: a direct, single-base fluorescent readout is possible per channel (though conferring less specificity), or multi-base encodings with one channel per base (see hyperspectral encoding).

### Combinatorial Sequencing Competitors

Combinatorial sequencing competitors consist of a pool of competitors corresponding to each combinatorial read position. For a given position, competitor oligos contain the competitor-specific sequence complementary to the read out oligo competitor-specific sequence, as well as sequence complementary to the read out sequence itself, plus 1-5 “N” bases (typically 3) to confer additional specificity to as much of the barcode and fluorophore oligo component as possible (these bases are “N” as the barcode sequence, and thus the fluorophore oligo sequence, is not known).

### Example 2

### Exemplary Sequences of Barcodes, Probes, and Competitors

```
              “qc”
          “barcode”: “ACCTATCCTTCCTA”,
          “toehold”: “ACTAGAATTCCACG”,
          “detection”: “/5ATTO488/ACTAGAATTCCACGACCTATCCTTCCTA”,
          “competitor”: “TAGGAAGGATAGGTCGTGGAATTCTAGT”
      “sequential”:
“fluor”:
              “A”: “ACTAAGCA”.
              “T”: “ACTAAGGT”,
              “C”: “ACTAACAC”
              “G”: “ACTAGTTG”
            “OR”:
              “OR1”:
                  “reading”: “/5Phos/TTACAATTTGA”,
                  “toehold”: “ATCCCACCCTCTACCA”,
                  “readout”: “/5Phos/TTACAATTTGAATCCCACCCTCTACCA”,
                  “competitor_A”: “TGGTAGAGGGTGGGATTCAAATTGTAATGCTTAGT”,
                  “competitor_T”: “TGGTAGAGGGTGGGATTCAAATTGTAAACCTTAGT”,
                  “competitor_C”: “TGGTAGAGGGTGGGATTCAAATTGTAAGTGTTAGT”,
                  “competitor_G”: “TGGTAGAGGGTGGGATTCAAATTGTAACAACTAGT”
              “OR2”:
                  “reading”: “/5Phos/ACTAATATCGA”,
                  “toehold”: “CTCTACCAACACCTCC”.
                  “readout”: “/5Phos/ACTAATATCGACTCTACCAACACCTCC”,
                  “competitor_A”: “GGAGGTGTTGGTAGAGTCGATATTAGTTGCTTAGT”,
                  “competitor_T”: “GGAGGTGTTGGTAGAGTCGATATTAGTACCTTAGT”,
                  “competitor_C”: “GGAGGTGTTGGTAGAGTCGATATTAGTGTGTTAGT”,
                  “competitor_G”: “GGAGGTGTTGGTAGAGTCGATATTAGTCAACTAGT”
              “OR3”:
                  “reading”: “/5Phos/CACTTTAATCT”,
                  “toehold”: “ACCACCCACCTACTCT”,
                  “readout”: “/5Phos/CACTTTAATCTACCACCCACCTACTCT”,
                  “competitor_A”: “AGAGTAGGTGGGTGGTAGATTAAAGTGTGCTTAGT”,
                  “competitor_T”: “AGAGTAGGTGGGTGGTAGATTAAAGTGACCTTAGT”,
                  “competitor_C”: “AGAGTAGGTGGGTGGTAGATTAAAGTGGTGTTAGT”,
                  “competitor_G”: “AGAGTAGGTGGGTGGTAGATTAAAGTGCAACTAGT”
              “OR4”:
                  “reading”: “/5Phos/GAAATCAAATC”,
                  “toehold”: “CCTCTTCCAACAACCC”,
                  “readout”: “/5Phos/GAAATCAAATCCCTCTTCCAACAACCC”,
                  “competitor_A”: “GGGTTGTTGGAAGAGGGATTTGATTTCTGCTTAGT”,
                  “competitor_T”: “GGGTTGTTGGAAGAGGGATTTGATTTCACCTTAGT”,
                  “competitor_C”: “GGGTTGTTGGAAGAGGGATTTGATTTCGTGTTAGT”,
                  “competitor_G”: “GGGTTGTTGGAAGAGGGATTTGATTTCCAACTAGT”
              “OR5”:
                  “reading”: “/5Phos/TGTGATATAAC”,
                  “toehold”: “ACACCACTACTCCTCC”
                  “readout”: “/5Phos/TGTGATATAACACACCACTACTCCTCC”,
                  “competitor_A”: “GGAGGAGTAGTGGTGTGTTATATCACATGCTTAGT”,
                  “competitor_T”: “GGAGGAGTAGTGGTGTGTTATATCACAACCTTAGT”,
                  “competitor_C”: “GGAGGAGTAGTGGTGTGTTATATCACAGTGTTAGT”,
                  “competitor_G”: “GGAGGAGTAGTGGTGTGTTATATCACACAACTAGT”
              “OR6”:
                  “reading”: “/5Phos/ATGTTACTAAG”,
                  “toehold”: “CTACCTCCCAATCCAC”,
                  “readout”: “/5Phos/ATGTTACTAAGCTACCTCCCAATCCAC”
                  “competitor_A”: “GTGGATTGGGAGGTAGCTTAGTAACATTGCTTAGT”,
                  “competitor_T”: “GTGGATTGGGAGGTAGCTTAGTAACATACCTTAGT”,
                  “competitor_C”: “GTGGATTGGGAGGTAGCTTAGTAACATGTGTTAGT”,
                  “competitor_G”: “GTGGATTGGGAGGTAGCTTAGTAACATCAACTAGT”
              “OR7”:
                  “reading”: “/5Phos/GTCGAATATAT”,
                  “toehold”: “TCCTACATCACCACCC”.
                  “readout”: “/5Phos/GTCGAATATATTCCTACATCACCACCC”,
                  “competitor_A”: “GGGTGGTGATGTAGGAATATATTCGACTGCTTAGT”,
                  “competitor_T”: “GGGTGGTGATGTAGGAATATATTCGACACCTTAGT”,
                  “competitor_C”: “GGGTGGTGATGTAGGAATATATTCGACGTGTTAGT”,
                  “competitor_G”: “GGGTGGTGATGTAGGAATATATTCGACCAACTAGT”
              “OR8”:
                  “reading”: “/5Phos/TCCTAGTTAAT”,
                  “toehold”: “CACCATTCACACTCCC”,
                  “readout”: “/5Phos/TCCTAGTTAATCACCATTCACACTCCC”,
                  “competitor_A”: “GGGAGTGTGAATGGTGATTAACTAGGATGCTTAGT”,
                  “competitor_T”: “GGGAGTGTGAATGGTGATTAACTAGGAACCTTAGT”,
                  “competitor_C”: “GGGAGTGTGAATGGTGATTAACTAGGAGTGTTAGT”,
                  “competitor_G”: “GGGAGTGTGAATGGTGATTAACTAGGACAACTAGT”
              “OR9”:
                  “reading”: “/5Phos/CGAAGATATTA”,
                  “toehold”: “ACCCACTTAACCTCCC”,
                  “readout”: “/5Phos/CGAAGATATTAACCCACTTAACCTCCC”,
                  “competitor_A”: “GGGAGGTTAAGTGGGTTAATATCTTCGTGCTTAGT”,
                  “competitor_T”: “GGGAGGTTAAGTGGGTTAATATCTTCGACCTTAGT”,
                  “competitor_C”: “GGGAGGTTAAGTGGGTTAATATCTTCGGTGTTAGT”,
                  “competitor_G”: “GGGAGGTTAAGTGGGTTAATATCTTCGCAACTAGT”
              “OR10”:
                  “reading”: “/5Phos/AGATCATCTTA”,
                  “toehold”: “CCAACTCCACCTCTAC”
                  “readout”: “/5Phos/AGATCATCTTACCAACTCCACCTCTAC”,
                  “competitor_A”: “GTAGAGGTGGAGTTGGTAAGATGATCTTGCTTAGT”,
                  “competitor_T”: “GTAGAGGTGGAGTTGGTAAGATGATCTACCTTAGT”,
                  “competitor_C”: “GTAGAGGTGGAGTTGGTAAGATGATCTGTGTTAGT”,
                  “competitor_G”: “GTAGAGGTGGAGTTGGTAAGATGATCTCAACTAGT”
              “OR11”:
                  “reading”: “/5Phos/GCATATTCATA”,
                  “toehold”: “CAACTCCATCCCTCAC”,
                  “readout”: “/5Phos/GCATATTCATACAACTCCATCCCTCAC”,
                  “competitor_A”: “GTGAGGGATGGAGTTGTATGAATATGCTGCTTAGT”,
                  “competitor_T”: “GTGAGGGATGGAGTTGTATGAATATGCACCTTAGT”,
                  “competitor_C”: “GTGAGGGATGGAGTTGTATGAATATGCGTGTTAGT”,
                  “competitor_G”: “GTGAGGGATGGAGTTGTATGAATATGCCAACTAGT”
              “OR12”:
                  “reading”: “/5Phos/AAGACTTATCT”,
                  “toehold”: “ACTCCCAACCATCCCT”,
                  “readout”: “/5Phos/AAGACTTATCTACTCCCAACCATCCCT”,
                  “competitor_A”: “AGGGATGGTTGGGAGTAGATAAGTCTTTGCTTAGT”,
                  “competitor_T”: “AGGGATGGTTGGGAGTAGATAAGTCTTACCTTAGT”,
                  “competitor_C”: “AGGGATGGTTGGGAGTAGATAAGTCTTGTGTTAGT”,
                  “competitor_G”: “AGGGATGGTTGGGAGTAGATAAGTCTTCAACTAGT”
      “combinatorial”:
          “RO1R1”:
              “reading”: “/5Phos/ATACACTAAAG”
              “toehold”: “CCCACTACCCTTACAC”,
              “competitor”: “GTGTAAGGGTAGTGGGCTTTAGTGTATNNN”,
              “readout”: “/5Phos/ATACACTAAAGCCCACTACCCTTACAC”
          “RO1R2”:
              “reading”: “/5Phos/CATACACTAAA”,
              “toehold”: “TTAACCACCACCTCCC”,
              “competitor”: “GGGAGGTGGTGGTTAATTTAGTGTATGNNN”,
              “readout”: “/5Phos/CATACACTAAATTAACCACCACCTCCC”
          “RO1R3”:
              “reading”: “/5Phos/(N:25252525)CATACACTAA”,
              “toehold”: “CTCCCTTCCACCAACA”,
              “competitor”: “TGTTGGTGGAAGGGAGTTAGTGTATGNNNN”,
              “readout”: “/5Phos/(N:25252525)CATACACTAACTCCCTTCCACCAACA”
          “RO2L1”:
              “reading”: “CAAAATCAATT”,
              “toehold”: “TAACCCACCCTCCCTA”,
              “competitor”: “NNNTTAGTGTATGNTAGGGAGGGTGGGTTA”,
              “readout”: “TAACCCACCCTCCCTACAAAATCAATT”
          “RO2L2”:
              “reading”: “AAAATCAATTC”,
              “toehold”: “CCTCCCATATCCAACC”,
              “competitor”: “NNNTTAGTGTATGNGGTTGGATATGGGAGG”,
              “readout”: “CCTCCCATATCCAACCAAAATCAATTC”
          “RO2L3”:
              “reading”: “AAATCAATTC(N:25252525)”,
              “toehold”: “ATCCTACCCACACCCT”,
              “competitor”: “NNNTTAGTGTATGNAGGGTGTGGGTAGGAT”,
              “readout”: “ATCCTACCCACACCCTAAATCAATTC(N:25252525)”
          “RO1L1”:
              “reading”: “ATACACTAAAG”,
              “toehold”: “TACACACCACCTCCTC”,
              “competitor”: “NNNTTAGTGTATGNGAGGAGGTGGTGTGTA”,
              “readout”: “TACACACCACCTCCTCATACACTAAAG”
          “RO1L2”:
              “reading”: “TACACTAAAGC”,
              “toehold”: “CTCAACCTCCCACCAT”,
              “competitor”: “NNNTTAGTGTATGNATGGTGGGAGGTTGAG”,
              “readout”: “CTCAACCTCCCACCATTACACTAAAGC”
          “RO1L3”:
              “reading”: “ATACACTAAAGC(N:25252525)”,
              “toehold”: “ACCCTTCCTCACACCA”,
              “competitor”: “NNNTTAGTGTATGNTGGTGTGAGGAAGGGT”,
              “readout”: “ACCCTTCCTCACACCAATACACTAAAGC(N:25252525)”
          “RO2R1”:
              “reading”: “/5Phos/CAAAATCAATT”
              “toehold”: “CACCTCATCCACCACT”
              “competitor”: “AGTGGTGGATGAGGTGAATTGATTTTGNNN”,
              “readout”: “/5Phos/CAAAATCAATTCACCTCATCCACCACT”
          “RO2R2”:
              “reading”: “/5Phos/CCAAAATCAAT”
              “toehold”: “TTCTCCCACCCAACAC”,
              “competitor”: “GTGTTGGGTGGGAGAAATTGATTTTGGNNN”,
              “readout”: “/5Phos/CCAAAATCAATTTCTCCCACCCAACAC”
          “RO2R3”:
              “reading”: “/5Phos/(N:25252525)CCAAAATCAA”,
              “toehold”: “CCCACCTACCAATCCT”,
              “competitor”: “AGGATTGGTAGGTGGGTTGATTTTGGNNNN”,
              “readout”: “/5Phos/(N:25252525)CCAAAATCAACCCACCTACCAATCCT”
```

What is claimed is:

1. A method of sequencing by competitive annealing and ligation to determine a sequence of a target nucleic acid, the method comprising performing one or more sequencing cycles, each cycle comprising:
(a) contacting the target nucleic acid with a read oligonucleotide and a set of fluorescently labeled decoding probes, wherein the read oligonucleotide comprises a first complementarity region that is complementary to a reading sequence on the target nucleic acid, and wherein each decoding probe comprises a second complementarity region that is complementary to a probe binding site on the target nucleic acid;
(b) ligating the read oligonucleotide to one of the decoding probes of the set of fluorescently labeled decoding probes to generate a fluorescent ligation product, wherein the ligation only occurs when the read oligonucleotide and the decoding probe bind to adjacent sequences on the target nucleic acid and both the read oligonucleotide and the decoding probe have sequences that are exactly complementary to the sequence of the target nucleic acid;
(c) removing unligated probes;
(d) imaging the fluorescent ligation product to detect the fluorescent label of the decoding probe that ligated to the read oligonucleotide, wherein the fluorescent label identifies a nucleotide of the sequence of the target nucleic acid; and
(e) removing the fluorescent ligation product from the target nucleic acid by binding a competitor oligonucleotide to the ligation product, wherein the competitor oligonucleotide comprises a third complementarity region comprising a sequence that is complementary to the reading sequence on the target nucleic acid, wherein the fluorescent ligation product dissociates from the target nucleic acid.

2. The method of claim 1, wherein the set of fluorescently labeled decoding probes comprises:
a first probe encoding a guanine, wherein the first probe comprises a first fluorescent label,
a second probe encoding an adenine, wherein the second probe comprises a second fluorescent label,
a third probe encoding a cytosine, wherein the third probe comprises a third fluorescent label, and
a fourth probe encoding a thymine, wherein the fourth probe comprises a fourth fluorescent label.

3. The method of claim 1, wherein each fluorescently labeled decoding probe encodes 1 to 3 bases adjacent to a ligation junction where the read oligonucleotide is ligated to the fluorescently labeled decoding probe, wherein fluorescently labeled decoding probes encoding different sequences of bases comprise different fluorescent labels.

4. The method of claim 1, wherein the read oligonucleotide ranges in length from 8 to 11 nucleotides.

5. The method of claim 1, wherein the read oligonucleotide has a melting temperature ranging from 17° C. to 20° C.

6. The method of claim 1, wherein the competitor oligonucleotide further comprises a fourth complementarity region comprising a sequence that is complementary to at least a portion of the probe binding site.

7. The method of claim 1, wherein the fourth complementarity region of the competitor oligonucleotide comprises a sequence that is fully complementary to the entire probe binding site on the target nucleic acid.

8. The method of claim 1, wherein the competitor oligonucleotide further comprises a fifth complementarity region comprising a sequence that is complementary to a competitor-specific complementary site adjacent to the reading sequence on the target nucleic acid.

9. The method of claim 1, wherein the competitor-specific complementary site ranges in length from 2 nucleotides to 16 nucleotides.

10. The method of claim 1, wherein the read oligonucleotide further comprises a competitor-specific complementary sequence.

11. The method of claim 1, wherein the competitor-specific complementary sequence of the read oligonucleotide ranges in length from 2 nucleotides to 16 nucleotides.

12. The method of claim 1, wherein said removing further comprises washing the target nucleic acid, wherein the ligation product diffuses away from the target nucleic acid.

13. The method of claim 1, wherein for sequencing cycles following an initial sequencing cycle, the competitor oligonucleotide used in a previous cycle of sequencing is present during one or more subsequent cycles of sequencing.

14. The method of claim 1 or 2, wherein the sequence of the read oligonucleotide for a current cycle of sequencing is optimized to minimize cross-hybridization with read oligonucleotides for other sequencing cycles.

15. The method of claim 1 or 2, wherein the sequences of the fluorescently labeled decoding probes for a current cycle of sequencing are optimized to minimize cross-hybridization with the fluorescently labeled decoding probes for other sequencing cycles.

16. The method of claim 1, wherein multiple read oligonucleotides, sets of fluorescently labeled decoding probes, and competitor oligonucleotides having specificity for different target nucleic acids are used to sequence a plurality of different target nucleic acids simultaneously or sequentially.

17. The method of claim 3, wherein the competitor oligonucleotides remove ligation products from a previous round of sequencing from different target nucleic acids than target nucleic acids currently undergoing steps (a) or (b) of a sequencing cycle.

18. The method of claim 3, wherein the competitor oligonucleotides remove ligation products from a previous round of sequencing from the same target nucleic acids currently undergoing steps (a) or (b) of a sequencing cycle.

19. The method of claim 1, wherein the competitor oligonucleotide is a round-specific competitor oligonucleotide comprising a fourth complementarity region comprising a sequence that is complementary to the reading sequence for the next cycle of sequencing.

20. The method of claim 1, wherein sequencing reads are in a 5′ to 3′ forward direction or a 3′ to 5′ reverse direction.

21. The method of claim 7, wherein each fluorescently labeled decoding probe has a fluorophore modification at the 5′ end and each read oligonucleotide has a phosphate at the 5′ end for sequencing reads in the forward direction.

22. The method of claim 7, wherein each fluorescently labeled decoding probe has a phosphate at the 5′ end and a fluorophore modification at the 3′ end for sequencing reads in the reverse direction.

23. The method of claim 1, wherein the fluorescent labels are fluorescent dyes or quantum dots.

24. The method of any one of claims 1-17, wherein sequencing is performed with sequential encoding.

25. The method of claim 8, wherein each read oligonucleotide comprises a unique sequential orthogonal readout sequence and a unique adjacent competitor-specific complementary sequence for each cycle of sequencing.

26. The method of claim 9, wherein the unique sequential orthogonal readout sequence ranges in length from 8 nucleotides to 11 nucleotides, and the unique adjacent competitor-specific complementary sequence ranges in length from 2 nucleotides to 16 nucleotides.

27. The method of claim 9 or 10, wherein each competitor oligonucleotide comprises a sequence that is complementary to the unique sequential orthogonal readout sequence and the unique adjacent competitor-specific complementary sequence of the read oligonucleotide and at least a portion of the sequence of the fluorescently labeled decoding probe for each cycle of sequencing.

28. The method of claim 9 or 10, wherein the sequence of the competitor oligonucleotide has partial complementarity or full complementarity to the sequence of the fluorescently labeled decoding probe.

29. The method of any one of claims 1-17, wherein sequencing is performed with combinatorial encoding.

30. The method of claim 7, wherein multiple read oligonucleotides are used for sequencing, wherein each read oligonucleotide comprises a first complementarity region comprising a combinatorial readout sequence that is complementary to a reading sequence at a separate combinatorial read position on the target nucleic acid, wherein the reading sequence at each separate position on the target nucleic is adjacent to a probe binding site.

31. The method of claim 7, wherein each read oligonucleotide further comprises a competitor-specific complementary sequence adjacent to the reading sequence.

32. The method of claim 7, wherein the competitor-specific complementary sequence is not complementary to the fluorescently labeled decoding probe.

33. The method of claim 9, wherein the reading sequence ranges in length from 8 nucleotides to 11 nucleotides,

34. The method of claim 9, wherein the competitor-specific complementary sequence ranges in length from 2 nucleotides to 16 nucleotides.

35. The method of claim 9 or 10, wherein for each separate combinatorial read position, the competitor oligonucleotide comprises a sequence that is complementary to the combinatorial readout sequence and the competitor-specific complementary sequence of the read oligonucleotide and at least a portion of the sequence of the fluorescently labeled decoding probe for each cycle of sequencing.

36. The method of any one of claims 29-35, wherein the combinatorial encoding uses a hamming code.

37. A method for in situ gene sequencing of a target nucleic acid in a cell in an intact tissue, the method comprising:
(a) contacting a fixed and permeabilized intact tissue with at least a pair of oligonucleotide primers under conditions to allow for specific hybridization,
wherein the pair of primers comprise a first oligonucleotide and a second oligonucleotide;
wherein each of the first oligonucleotide and the second oligonucleotide comprises a first complementarity region, a second complementarity region sequence, and a third complementarity region; wherein the second oligonucleotide further comprises a barcode sequence;
wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid;
(b) adding ligase to ligate the second oligonucleotide and generate a closed nucleic acid circle;
(c) performing rolling circle amplification in the presence of a nucleic acid molecule, wherein the performing comprises using the second oligonucleotide as a template and the first oligonucleotide as a primer for a polymerase to form one or more amplicons;
(d) embedding the one or more amplicons in the presence of hydrogel subunits to form one or more hydrogel-embedded amplicons;
(e) sequencing the one or more amplicons according to the method of any one of claims 1-36.

38. The method of claim 37, further comprising preincubating the tissue sample with the polymerase for a sufficient time to allow uniform diffusion of the polymerase throughout the tissue before performing the rolling circle amplification.

39. The method of claim 37 or 38, wherein said imaging is performed in presence of an anti-fade buffer comprising an antioxidant.

40. The method of claim 39, wherein the anti-fade buffer comprises Trolox (6-hydroxy-2,5,7,8-tetramethylchroman-2-carboxylic acid) and Trolox-quinone.

41. The method of any one of claims 37-40, wherein the cell is present in a population of cells.

42. The method of claim 41, wherein the population of cells comprises a plurality of cell types.

43. The method of any one of claims 37-42, wherein the contacting the fixed and permeabilized intact tissue comprises hybridizing the primers to the same target nucleic acid.

44. The method of any one of claims 37-43, wherein the target nucleic acid is RNA or DNA.

45. The method of claim 44, wherein the RNA is mRNA.

46. The method of any one of claims 37-45, wherein the second oligonucleotide comprises a padlock probe.

47. The method of any one of claims 37-46, wherein the first complementarity region of the first oligonucleotide has a length of 19-25 nucleotides.

48. The method of any one of claims 37-47, wherein the second complementarity region of the first oligonucleotide has a length of 6 nucleotides.

49. The method of any one of claims 37-48, wherein the third complementarity region of the first oligonucleotide has a length of 6 nucleotides.

50. The method of any one of claims 37-49, wherein the first complementarity region of the second oligonucleotide has a length of 6 nucleotides.

51. The method of any one of claims 37-50, wherein the second complementarity region of the second oligonucleotide has a length of 19-25 nucleotides.

52. The method of any one of claims 37-51, wherein the third complementarity region of the second oligonucleotide has a length of 6 nucleotides.

53. The method of any one of claims 37-52, wherein the first complementarity region of the second oligonucleotide comprises the 5′ end of the second oligonucleotide.

54. The method of any one of claims 37-53, wherein the third complementarity region of the second oligonucleotide comprises the 3′ end of the second oligonucleotide.

55. The method of any one of claims 37-54, wherein the first complementarity region of the second oligonucleotide is adjacent to the third complementarity region of the second oligonucleotide.

56. The method of any one of claims 37-55, wherein the barcode sequence of the second oligonucleotide provides barcoding information for identification of the target nucleic acid.

57. The method of any one of claims 37-56, wherein the contacting the fixed and permeabilized intact tissue comprises hybridizing a plurality of oligonucleotide primers having specificity for different target nucleic acids.

58. The method of any one of claims 37-57, wherein the second oligonucleotide is provided as a closed nucleic acid circle, and the step of adding ligase is omitted.

59. The method of any of claims 37-58, wherein the melting temperature (Tₘ) of oligonucleotides is selected to minimize ligation in solution.

60. The method of any one of claims 37-59, wherein the adding ligase comprises adding a DNA ligase.

61. The method of any one of claims 37-60, wherein the nucleic acid molecule comprises an amine-modified nucleotide.

62. The method of claim 61, wherein the amine-modified nucleotide comprises an acrylic acid N-hydroxysuccinimide moiety modification.

63. The method of any one of claims 37-62, wherein the embedding comprises copolymerizing the one or more amplicons with acrylamide.

64. The method of any one of claims 37-63, wherein the embedding comprises clearing the one or more hydrogel-embedded amplicons wherein the target nucleic acid is substantially retained in the one or more hydrogel-embedded amplicons.

65. The method of claim 64, wherein the clearing comprises substantially removing a plurality of cellular components from the one or more hydrogel-embedded amplicons.

66. The method of claim 64 or 65, wherein the clearing comprises substantially removing lipids or proteins, or a combination thereof from the one or more hydrogel-embedded amplicons.

67. The method of any one of claims 37-66, wherein the contacting the one or more hydrogel-embedded amplicons comprises eliminating error accumulation as sequencing proceeds.

68. The method of any one of claims 37-67, wherein the imaging comprises imaging the one or more hydrogel-embedded amplicons using confocal microscopy, two-photon microscopy, light-field microscopy, intact tissue expansion microscopy, and/or CLARITY™-optimized light sheet microscopy (COLM).

69. The method of any one of claims 37-68, wherein the intact tissue is a thin slice.

70. The method of claim 69, wherein the intact tissue has a thickness of 5-20 μm.

71. The method of claim 68 or 69, wherein the contacting the one or more hydrogel-embedded amplicons occurs four times or more.

72. The method of any one of claims 37-71, wherein the intact tissue is a thick slice.

73. The method of claim 72, wherein the intact tissue has a thickness of 20-200 μm.

74. The method of claim 71 or 72, wherein the contacting the one or more hydrogel-embedded amplicons occurs six times or more.

75. The method of any one of claims 37-74, wherein the ligation of the third oligonucleotide and the fourth oligonucleotide is performed in presence of a polyethylene glycol polymer.

76. The method of claim 75, wherein the PEG polymer is PEG 6000.

77. A method of screening a candidate agent to determine whether the candidate agent modulates gene expression of a nucleic acid in a cell in an intact tissue, the method comprising performing the method of any one of claims 37-76 to determine the gene sequence of the target nucleic acid in the cell in the intact tissue, and
detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

78. The method of claim 77, wherein the detecting comprises performing flow cytometry;
sequencing; probe binding and electrochemical detection; pH alteration; catalysis induced by enzymes bound to DNA tags; quantum entanglement; Raman spectroscopy; terahertz wave technology; and/or scanning electron microscopy.

79. The method of claim 78, wherein the flow cytometry is mass cytometry or fluorescence-activated flow cytometry.

80. The method of any one of claims 77-79, wherein the detecting comprises performing microscopy, scanning mass spectrometry, or other imaging techniques

81. The method of any one of claims 77-80, wherein the detecting comprises detecting a signal.

82. The method of claim 81, wherein the signal is a fluorescent signal.

83. A system, comprising:
a fluidics device, and
a processor unit configured to perform the method of any one of claims 1-82.

84. The system of claim 83, further comprising an imaging chamber.

85. The system of claim 83 or 84, further comprising a pump.
